(19) United States
(12) Patent Application Publication
(10) Pub. No.: US 2026/0308841 A1
(43) Pub. Date: Oct. 8, 2026

(54) DISPERSE DYES FOR DYEING POLYESTER FIBRES

(71) Applicant: University of Leeds, Leeds (GB)

(72) Inventors: Richard Simon BLACKBURN, Leeds, West Yorkshire (GB); Christopher Mark RAYNER, Leeds, West Yorkshire (GB); Nathaniel Beaumont CROMPTON, Leeds, West Yorkshire (GB); Harrison George OATES, Leeds, West Yorkshire (GB)

(73) Assignee: University of Leeds, Leeds (GB)

(21) Appl. No.: 19/481,396

(22) PCT Filed: May 3, 2024
(86) PCT No.: PCT/GB2024/051181

§ 371 Date: Nov. 4, 2025

(30) Foreign Application Priority Data
May 4, 2023 (GB) 2306595.6

(51) Int. Cl. D06P 3/54; C07C 213/08; C07C 217/92; C07C 221/00; C07C 225/34; C07C 225/36; C07C 245/08; C07D 209/58; C07D 295/135; C09B 1/28; C09B 1/32; C09B 1/515; C09B 5/24; C09B 29/00; C09B 51/00; C09B 67/14; D06P 5/13; D06P 5/20

(52) U.S. Cl. CPC D06P 3/54; C07C 213/08; C07C 217/92; C07C 221/00; C07C 225/34; C07C 225/36; C07C 245/08; C07D 209/58; C07D 295/135; C09B 1/285; C09B 1/325; C09B 1/5155; C09B 5/2472; C09B 29/00; C09B 51/00; C09B 67/0017; D06P 5/137; D06P 5/2077; C07C 2603/24

(57) ABSTRACT

The present invention relates to compounds of formula (I): wherein R¹ is a dye, and L′, R² and R³ are as defined herein. The compounds of the invention reversibly convert from a hydrophobic form to a hydrophilic form upon contact with water and carbon dioxide. The present invention also relates to methods of dyeing fibres, and methods of decolouring dyed fibres. The method is particularly useful for dyeing polyester fibres.

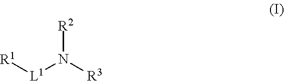

The present invention relates to compounds of Formula (I), wherein the compounds comprise a dye. The compounds of the invention reversibly convert from a hydrophobic form to a hydrophilic form upon contact with water and carbon dioxide. The present invention also relates to methods of dyeing fibres, and methods of decolouring dyed fibres. The method is particularly useful for dyeing polyester fibres.

### BACKGROUND

A large proportion of the textile products produced in the world today comprise polyesters. In particular, poly(ethylene terephthalate) (PET) is the most widely produced fibre and accounted for 54% (57×10⁶ T) of the 113×10⁶ T world textile fibre demand in 2021. The outstanding success and enduring popularity of PET fibres can be attributed to their generally excellent textile characteristics and high chemical resistance, coupled with the ability of polyester fibre to be manufactured in virtually any physical form as required for different applications, which include blending with other types of fibre.

Polyester fibres are dyed almost exclusively using disperse dyes, which furnish a wide shade gamut and display generally very good fastness properties on polyester. Disperse dyes belong to several chemical classes, predominantly anthraquinonoid (AQ), and azo (AZ), as exemplified by C.I. Disperse Red 60 and C.I. Disperse Blue 165, respectively.

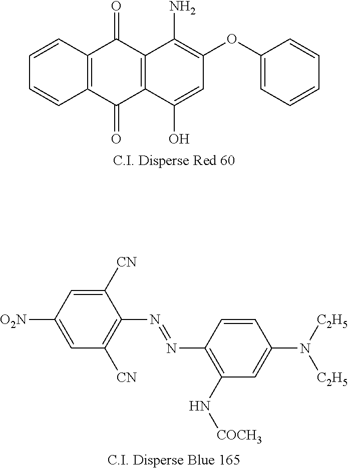 [SMILES: Nc1c(Oc2ccccc2)cc(O)c2c1C(=O)c1ccccc1C2=O ; CCN(CC)c1ccc(/N=N/c2c(C#N)cc([N+](=O)[O-])cc2C#N)c(NC(C)=O)c1]

Conventional disperse dyes have fixed physical properties which can lead to significant limitations in their use. For example, disperse dyes are hydrophobic, and as such have low aqueous solubility. The solubility of disperse dyes is greatly increased in the presence of auxiliary chemicals such as commercial dispersing agents, a feature that forms the basis of both the finishing process that is used to prepare commercial forms of the dye and the application method that is employed in their application to polyester fibres under aqueous immersion processes. Commercial disperse dyes typically comprise up to 60% by mass dispersing agent, adding considerably to the cost of the dyes and their environmental burden. Dispersing agents (examples include lignin sulfonates, formaldehyde polycondensates, or arylsulfonic acids) are not particularly environmentally friendly.

Polyester is also hydrophobic, and is dyed with hydrophobic disperse dyes in an aqueous medium. Therefore, many auxiliary agents are required in dyeing polyester, most significantly dispersing agents. Dispersing agents allow disperse dyes to form a dispersion in water, thereby disperse dyes can be used in a homogeneous dye liquor to dye polyester. However, in industry, the dye, water, and dispersing agents are not recycled, as after the dye liquor has been used, the remaining dispersing agent inhibits re-use. The treatment and replacement of dye and dispersing agents greatly adds to the economic cost, energy efficiency, and environmental impact of the overall dyeing process.

Disperse dyes are much more soluble in organic solvents than they are in water. However, organic solvents are ineffective for disperse dyeing because the dye has a greater affinity for the solvent than it does for the fibres. Consequently, when disperse dyes are applied from organic solvents low dye uptake onto the fibres is achieved so that the depth of shade that can be accessed using high temperature aqueous dyeing techniques cannot be replicated using organic solvent based dyeing liquors.

As discussed above, dyeing auxiliaries are commonly used to assist aqueous immersion dyeing processes. The assistance provided by a given dyeing auxiliary will typically relate to a specific aspect of dyeing, such as wetting, dye levelling, fibre protection, etc. Thus, many different types of dyeing auxiliary are commonly used in aqueous dye application, such as dispersants, sequestrants, lubricants, etc. For example, dyeing processes for polyester fibres typically utilise dispersing agents and surfactants which are added to the dyebath to aid dye dispersion and levelling, as discussed above. Also, the pH at which dyeing is performed is commonly adjusted so as to be within a defined range, such as slightly acidic (pH ~4.5-6.0), although selected disperse dyes are suitable for application at high pH (~pH 9.5). Because aqueous dyebaths routinely contain many auxiliaries often in large amounts, the wastewater generated during immersion dyeing is likely to contain a wide variety of auxiliary chemicals; indeed, many of the auxiliaries that are utilised in immersion dyeing (such as dispersing agents that are used in polyester dyeing) are intended to be removed from the dyed material at the end of dyeing, meaning that such chemicals will be present in the wastewater that ensues from dyeing processes. Although many strategies have been explored for treating dyeing effluent that contains residual dyeing auxiliaries, no single treatment method is effective for all auxiliaries or types of dye/fibre system. An additional advantage of certain embodiments of the current invention is that it is possible to reduce the number and amount of dyeing auxiliaries that are used in dyeing processes, thus offering savings in chemical costs as well as environmental advantages.

Therefore, there still exists a need for a dye that can change its physical properties in a manner that would enhance its effectiveness in a dyeing process. In particular, a dye for polyester, which can be reversibly converted between hydrophobic and hydrophilic forms.

### BRIEF SUMMARY OF THE DISCLOSURE

In a first aspect of the invention, there is provided a compound of Formula (I):

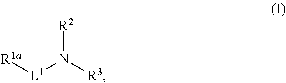

wherein:
R^{1a} is a dye, optionally substituted with a further L¹NR²R³ group;
L¹ is independently at each occurrence selected from —C₁₋₅-alkylene-X—C₀₋₅-alkylene- or —C₀₋₅-alkylene-X—C₁₋₅-alkylene-, wherein X is absent or selected from C₃₋₆-cycloalkylene, 4- to 7-membered heterocyclyl, 5- to 10-membered heteroaryl and phenyl;
R² is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
L¹ and R² together with the nitrogen to which they are attached form a 3- to 6-membered heterocyclyl optionally substituted, where chemically possible, by 1 to 3 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein the heterocyclyl has 1 to 2 additional heteroatom ring vertices selected from the group consisting of O, N, and S;
R³ is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
R² and R³ together with the nitrogen to which they are attached form a 3- to 6-membered heterocyclyl;
R⁴ is independently at each occurrence selected from: C₃₋₆-cycloalkyl, 4- to 7-membered heterocyclyl, 5- to 10-membered heteroaryl and phenyl;
R⁵ is independently at each occurrence selected from: OR⁶, CO₂R⁶, NR⁶R⁷, CONR⁶R⁷, SR⁶, and S(O)₂NR⁶R⁷;
R⁶ is independently at each occurrence selected from H, C₁₋₆-alkyl, C₃₋₆-cycloalkyl, 4- to 7-membered heterocyclyl;
R⁷ is independently at each occurrence selected from H, C₁₋₆-alkyl, C₃₋₆-cycloalkyl, 4- to 7-membered heterocyclyl, C(O)R⁸, S(O)₂R⁸; and
R⁸ is independently at each occurrence C₁₋₆-alkyl, C₃₋₆-cycloalkyl, 4- to 7-membered heterocyclyl, 5- to 10-membered heteroaryl and phenyl;
wherein any R¹-R⁸ group that is an alkyl, alkylene, cycloalkyl, heterocyclyl, phenyl or heteroaryl may be optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl;
wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

In this embodiment, it may be that R¹ is a disperse dye.

The inventors have found that the compounds of the invention are switchable compounds. The compounds contain one or more amines and switch between a neutral form and a charged form (ammonium ion or carbamate ion) in response to a selected trigger. The charged form provides an ionic solute at moderate temperatures, e.g., at room temperature. The trigger to change from neutral form to charged form may be exposure of the neutral form to carbon dioxide, carbon disulfide, or carbonyl sulfide in the presence of water (see FIG. 1). Preferably, the trigger to change from neutral form to charged form is exposure of the neutral form to carbon dioxide in the presence of water. The compounds of the invention are not only switchable, but reversibly so. Thus, removal of the trigger, e.g. removing CO₂, causes the charged form to switch to the neutral form (see FIG. 2). This could be achieved, for example, by raising the temperature of the dyebath solution or by purging with a stream of inert gas.

The inventors have found that the compounds of the invention can be used for dyeing hydrophobic fibres, such as polyester fibres, using this property of switching the physical properties of the compounds (e.g., polarity). A solution of the compound in its ionic form is used to wet the fibre. Then the compound is switched to its neutral form and the compound's affinity for the solution goes down and its affinity for the fibre increases, causing the compound to preferentially associate with the fibre and thus dyeing it. If the compound is switched to its ionic form, the compound can be removed from the fibre. The invention therefore provides a switchable compound system that can be reversibly and readily switched between non-ionic and ionic forms by applying or removing a trigger such as CO₂. By heating the dye liquor with the fibres (e.g. polyester fibres), the CO₂ can be removed and the non-ionic form can be reformed towards the dyeing of the fibres (e.g. polyester fibres). Advantageously, this means that hydrophobic fibres, such as polyester fibres can be dyed with the compounds of the invention (e.g. comprising a disperse dye) in the absence of auxiliary chemicals (other than carbon dioxide, carbon disulfide, or carbonyl sulfide), most significantly in the absence of dispersing agents.

In a second aspect of the invention, there is provided a salt of Formula (II):

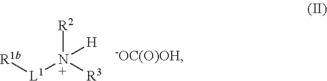

wherein:
R^{1b} is a dye optionally substituted with a L¹NR²R³ group or a further L¹NH⁺R²R³·⁻OC(O)OH group;
L¹ is independently at each occurrence selected from —C₁₋₅-alkylene-X—C₀₋₅-alkylene- or —C₀₋₅-alkylene-X—C₁₋₅-alkylene-, wherein X is absent or selected from C₃₋₆-cycloalkylene, 4- to 7-membered heterocyclyl, 5- to 10-membered heteroaryl and phenyl;
R² is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
L¹ and R² together with the nitrogen to which they are attached form a 3- to 6-membered heterocyclyl optionally substituted, where chemically possible, by 1 to 3 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein the heterocyclyl has 1 to 2 additional heteroatom ring vertices selected from the group consisting of O, N, and S;
R³ is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
R² and R³ together with the nitrogen to which they are attached form a 3- to 6-membered heterocyclyl;
R⁴ is independently at each occurrence selected from: C₃₋₆-cycloalkyl, 4- to 7-membered heterocyclyl, 5- to 10-membered heteroaryl and phenyl;
R⁵ is independently at each occurrence selected from: OR⁶, CO₂R⁶, NR⁶R⁷, CONR⁶R⁷, SR⁶, and S(O)₂NR⁶R⁷;
R⁶ is independently at each occurrence selected from H, C₁₋₆-alkyl, C₃₋₆-cycloalkyl, 4- to 7-membered heterocyclyl;
R⁷ is independently at each occurrence selected from H, C₁₋₆-alkyl, C₃₋₆-cycloalkyl, 4- to 7-membered heterocyclyl, C(O)R⁸, S(O)₂R⁸; and
R⁸ is independently at each occurrence C₁₋₆-alkyl, C₃₋₆-cycloalkyl, 4- to 7-membered heterocyclyl, 5- to 10-membered heteroaryl and phenyl;
wherein any R¹-R⁸ group that is an alkyl, alkylene, cycloalkyl, heterocyclyl, phenyl or heteroaryl may be optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

In this embodiment, it may be that R¹ is a disperse dye.

In a third aspect of the invention, there is provided a carbamate of Formula (III):

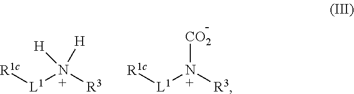

wherein:
R^{1c} is a dye, optionally substituted with a L¹NR²R³ group, a further L¹N⁺H₂R³ group and/or a further L¹N(CO₂⁻)R³ group;
R² is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
L¹ is independently at each occurrence selected from —C₁₋₅-alkylene-X—C₀₋₅-alkylene- or —C₀₋₅-alkylene-X—C₁₋₅-alkylene-, wherein X is absent or selected from C₃₋₆-cycloalkylene, 4- to 7-membered heterocyclyl, 5- to 10-membered heteroaryl and phenyl; or
L¹ and R² together with the nitrogen to which they are attached form a 3- to 6-membered heterocyclyl optionally substituted, where chemically possible, by 1 to 3 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein the heterocyclyl has 1 to 2 additional heteroatom ring vertices selected from the group consisting of O, N, and S;
R³ is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
R² and R³ together with the nitrogen to which they are attached form a 3- to 6-membered heterocyclyl;
R⁴ is independently at each occurrence selected from: C₃₋₆-cycloalkyl, 4- to 7-membered heterocyclyl, 5- to 10-membered heteroaryl and phenyl;
R⁵ is independently at each occurrence selected from: OR⁶, CO₂R⁶, NR⁶R⁷, CONR⁶R⁷, SR⁶, and S(O)₂NR⁶R⁷;
R⁶ is independently at each occurrence selected from H, C₁₋₆-alkyl, C₃₋₆-cycloalkyl, 4- to 7-membered heterocyclyl;
R⁷ is independently at each occurrence selected from H, C₁₋₆-alkyl, C₃₋₆-cycloalkyl, 4- to 7-membered heterocyclyl, C(O)R⁸, S(O)₂R⁸; and
R⁸ is independently at each occurrence C₁₋₆-alkyl, C₃₋₆-cycloalkyl, 4- to 7-membered heterocyclyl, 5- to 10-membered heteroaryl and phenyl;
wherein any R¹-R⁸ group that is an alkyl, alkylene, cycloalkyl, heterocyclyl, phenyl or heteroaryl may be optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

In this embodiment, it may be that R¹ is a disperse dye.

In a fourth aspect of the invention, there is provided a method of dyeing fibres, the method comprising the following steps:
a) subjecting the fibres to a dye liquor comprising water to provide the fibres wetted with the dye liquor;
b) heating the fibres wetted with the dye liquor to a temperature of from 90° C. to 180° C., to provide dyed fibres wetted with an exhausted dye liquor comprising water; and
c) removing the dyed fibres from the exhausted dye liquor,
wherein the dye liquor of step a) further comprises:
i) a compound of Formula (I) according to the first aspect of the invention and carbon dioxide;
ii) a salt of Formula (II) according to the second aspect of the invention, a carbamate of Formula (III) according to the third aspect of the invention, or mixtures thereof; or
iii) a salt of Formula (IV) according to the sixth aspect of the invention and a metal bicarbonate.

In embodiments, the dye liquor of step a) comprises water, and a salt of Formula (II), or a carbamate of Formula (III), or mixtures thereof.

In embodiments, the dye liquor of step a) comprises water, and a salt of Formula (II), or a carbamate of Formula (III), or mixtures thereof and the dye liquor of step a) is obtained by:
i) obtaining an aqueous dye liquor comprising water and a compound of Formula (I) as described herein; and
ii) exposing the aqueous dye liquor to carbon dioxide to form the dye liquor.

In embodiments, the dye liquor of step a) comprises water and a salt of Formula (II) and the dye liquor of step a) is obtained by:
i) obtaining an aqueous dye liquor comprising water and a salt of Formula (IV) as described herein; and
ii) exposing the aqueous dye liquor to a metal bicarbonate to form the dye liquor.

In embodiments, the dye liquor comprises water, and a salt of Formula (II). In other embodiments, the dye liquor comprises water, and a carbamate of Formula (III). Alternatively, it may be that the dye liquor comprises a mixture of both a salt of Formula (II) and a carbamate of Formula (III).

In a fifth aspect of the invention, there is provided a method of decolouring dyed fibres, wherein the fibres are dyed with a compound of Formula (I) as described herein, the method comprising exposing the dyed fibres to a liquid comprising carbon dioxide.

In this embodiment, it may be that in the compound of Formula (I), R¹ is a disperse dye.

In a sixth aspect of the invention, there is provided a salt of Formula (IV):

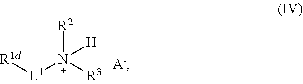

wherein:
R^{1d} is a dye, optionally substituted with a L¹NR²R³ group or a further L¹NH⁺R²R³·⁻A group;
A⁻ is an anionic counterion derived from a Brønsted acid;
L¹ is independently at each occurrence selected from —C₁₋₅-alkylene-X—C₀₋₅-alkylene- or —C₀₋₅-alkylene-X—C₁₋₅-alkylene-, wherein X is absent or selected from C₃₋₆-cycloalkylene, 4- to 7-membered heterocyclyl, 5- to 10-membered heteroaryl and phenyl;
R² is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
L¹ and R² together with the nitrogen to which they are attached form a 3- to 6-membered heterocyclyl optionally substituted, where chemically possible, by 1 to 3 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein the heterocyclyl has 0 to 2 additional heteroatom ring vertices selected from the group consisting of O, N, and S;
R³ is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
R² and R³ together with the nitrogen to which they are attached form a 3- to 6-membered heterocyclyl;
R⁴ is independently at each occurrence selected from: C₃₋₆-cycloalkyl, 4- to 7-membered heterocyclyl, 5- to 10-membered heteroaryl and phenyl;
R⁵ is independently at each occurrence selected from: OR⁶, CO₂R⁶, NR⁶R⁷, CONR⁶R⁷, SR⁶, and S(O)₂NR⁶R⁷;
R⁶ is independently at each occurrence selected from H, C₁₋₆-alkyl, C₃₋₆-cycloalkyl, 4- to 7-membered heterocyclyl;
R⁷ is independently at each occurrence selected from H, C₁₋₆-alkyl, C₃₋₆-cycloalkyl, 4- to 7-membered heterocyclyl, C(O)R⁸, S(O)₂R⁸; and
R⁸ is independently at each occurrence C₁₋₆-alkyl, C₃₋₆-cycloalkyl, 4- to 7-membered heterocyclyl, 5- to 10-membered heteroaryl and phenyl;
wherein any R¹-R⁸ group that is an alkyl, alkylene, cycloalkyl, heterocyclyl, phenyl or heteroaryl may be optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl;
wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

In an embodiment, R¹ is a disperse dye.

In a seventh aspect of the invention, there is provided a method of forming a salt of Formula (II) according to the second aspect of the invention, a carbamate of Formula (III) according to the third aspect of the invention, or a mixture thereof, the method comprising contacting a compound of Formula (I) according to the first aspect of the invention with carbon dioxide.

In an eighth aspect of the invention, there is provided a method of forming a salt of Formula (II) according to the second aspect of the invention, the method comprising contacting a salt of Formula (IV) according to the sixth aspect of the invention with a metal bicarbonate salt.

### The Compounds

The following embodiments apply to the compound of Formula (I), the salt of Formula (II), the carbamate of Formula (III), or the salt of Formula (IV). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

In embodiments, R^{1a} is a dye as defined herein. It may be that R^{1a} is a dye selected from a disperse dye, a solvent dye, a vat dye, a sulphur dye, a mordant dye, an acid dye, a direct dye and a reactive dye. In preferred embodiments, R^{1a} is a disperse dye as defined herein. It may be that R^{1a} is a disperse dye selected from the group consisting of azo, anthraquinone, nitro, aminoketone, methine, or naphthoquinone dyes. Preferably, R^{1a} is an azo dye or an anthraquinone dye.

In embodiments, R^{1a} is an azo dye. It may be that the azo dye is selected from the group consisting of: Disperse Red 1, Disperse Red 9, Disperse Red 17, Disperse Red 19, Disperse Red 25, Disperse Orange 3, Disperse Yellow 3, Solvent Yellow 14, and Solvent Orange 7. It may be that Ria is Disperse Red 1 (DR1).

In embodiments, R^{1a} is an anthraquinone dye. It may be that the anthraquinone dye is selected from the group consisting of: Solvent Orange 86, Disperse Blue 3, Disperse Blue 60, Disperse Blue 87, Disperse Violet 1, and Azoic Diazo 36. It may be that R^{1a} is Disperse Violet 1 (DV1).

In embodiments, R^{1a} is a nitro dye, e.g. Disperse Yellow 26.

It may be that R^{1a} is not substituted with a further L¹NR²R³ group.

In embodiments, R^{1b} is a dye as defined herein. It may be that R^{1b} is a dye selected from a disperse dye, a solvent dye, a vat dye, a sulphur dye, a mordant dye, an acid dye, a direct dye and a reactive dye. In preferred embodiments, R^{1b} is a disperse dye as defined herein. It may be that R^{1b} is a disperse dye selected from the group consisting of azo, anthraquinone, nitro, aminoketone, methine, or naphthoquinone dyes. Preferably, R^{1b} is an azo dye or an anthraquinone dye.

In embodiments, R^{1b} is an azo dye. It may be that the azo dye is selected from the group consisting of: Disperse Red 1, Disperse Red 9, Disperse Red 17, Disperse Red 19, Disperse Red 25, Disperse Orange 3, Disperse Yellow 3, Solvent Yellow 14, and Solvent Orange 7. It may be that R^{1b} is Disperse Red 1 (DR1).

In embodiments, R^{1b} is an anthraquinone dye. It may be that the anthraquinone dye is selected from the group consisting of: Solvent Orange 86, Disperse Blue 3, Disperse Blue 60, Disperse Blue 87, Disperse Violet 1, and Azoic Diazo 36. It may be that R^{1b} is Disperse Violet 1 (DV1).

In embodiments, R^{1b} is a nitro dye, e.g. Disperse Yellow 26.

It may be that R^{1b} is not substituted with a L¹NR²R³ group or a further L¹NH⁺R²R³·⁻OC(O)OH group.

In embodiments, R^{1c} is a dye as defined herein. It may be that R^{1c} is a dye selected from a disperse dye, a solvent dye, a vat dye, a sulphur dye, a mordant dye, an acid dye, a direct dye and a reactive dye. In preferred embodiments, R^{1c} is a disperse dye as defined herein. It may be that R^{1c} is a disperse dye selected from the group consisting of azo, anthraquinone, nitro, aminoketone, methine, or naphthoquinone dyes. Preferably, R^{1c} is an azo dye or an anthraquinone dye.

In embodiments, R^{1c} is an azo dye. It may be that the azo dye is selected from the group consisting of: Disperse Red 1, Disperse Red 9, Disperse Red 17, Disperse Red 19, Disperse Red 25, Disperse Orange 3, Disperse Yellow 3, Solvent Yellow 14, and Solvent Orange 7. It may be that R^{1c} is Disperse Red 1 (DR1).

In embodiments, R^{1c} is an anthraquinone dye. It may be that the anthraquinone dye is selected from the group consisting of: Solvent Orange 86, Disperse Blue 3, Disperse Blue 60, Disperse Blue 87, Disperse Violet 1, and Azoic Diazo 36. It may be that R^{1c} is Disperse Violet 1 (DV1).

In embodiments, R^{1c} is a nitro dye, e.g. Disperse Yellow 26.

It may be that R^{1c} is not substituted with an L¹NR²R³ group, a further L¹N⁺H₂R³ group and/or a further L¹N(CO₂⁻)R³ group.

In embodiments, R^{1d} is a dye as defined herein. It may be that R^{1d} is a dye selected from a disperse dye, a solvent dye, a vat dye, a sulphur dye, a mordant dye, an acid dye, a direct dye and a reactive dye. In preferred embodiments, R^{1d} is a disperse dye as defined herein. It may be that R^{1d} is a disperse dye selected from the group consisting of azo, anthraquinone, nitro, aminoketone, methine, or naphthoquinone dyes. Preferably, R^{1d} is an azo dye or an anthraquinone dye.

In embodiments, R^{1d} is an azo dye. It may be that the azo dye is selected from the group consisting of: Disperse Red 1, Disperse Red 9, Disperse Red 17, Disperse Red 19, Disperse Red 25, Disperse Orange 3, Disperse Yellow 3, Solvent Yellow 14, and Solvent Orange 7. It may be that R^{1d} is Disperse Red 1 (DR1).

In embodiments, R^{1d} is an anthraquinone dye. It may be that the anthraquinone dye is selected from the group consisting of: Solvent Orange 86, Disperse Blue 3, Disperse Blue 60, Disperse Blue 87, Disperse Violet 1, and Azoic Diazo 36. It may be that R^{1d} is Disperse Violet 1 (DV1).

In embodiments, R^{1d} is a nitro dye, e.g. Disperse Yellow 26.

It may be that R^{1d} is not substituted with an L¹NR²R³ group or a further L¹NH⁺R²R³·⁻A group.

In embodiments, L¹ is —C₁₋₁₀-alkylene-. It may be that L¹ is —C₁₋₆-alkylene-. It may be that L¹ is —C₂₋₈-alkylene-. It may be that L¹ is —C₃₋₅-alkylene-. It may be that L¹ is —C₁₋₃-alkylene-. Preferably, L¹ is —CH₂—.

It may be that L¹ is —C₁₋₁₀-alkylene-substituted with an oxo group. Where L¹ is substituted with an oxo group, it may be substituted at the carbon atom adjacent to R^{1a}, R^{1b}, R^{1c} or R^{1d}. Ideally, where L¹ is —C₁₋₁₀-alkylene-substituted with an oxo group, the —C₁₋₁₀-alkylene- is not substituted with an oxo group at the carbon atom adjacent to the terminal amino or ammonium group of Formulae (I)-(IV).

It may be that L¹ is —C(O)—C₁₋₉-alkylene. It may be that L¹ is —C(O)—C₂₋₆-alkylene. It may be that L¹ is

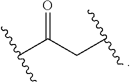 [SMILES: *CC(*)=O]

It may be that L¹ is

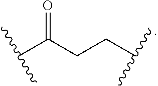 [SMILES: *CCC(*)=O]

It may be that L¹ is

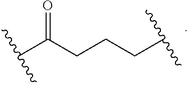 [SMILES: *CCCC(*)=O]

In embodiments L¹ is independently at each occurrence selected from —C₁₋₅-alkylene-X—C₀₋₅-alkylene- or —C₀₋₅-alkylene-X—C₁₋₅-alkylene-, wherein X is selected from C₃₋₆-cycloalkyl, 4- to 7-membered heterocyclyl, 5- to 10-membered heteroaryl and phenyl. Where X is 5- to 10-membered heteroaryl and phenyl, L¹ may be —C₀₋₅-alkylene-X—C₁₋₅-alkylene-.

In embodiments L¹ is independently at each occurrence —C₀₋₅-alkylene-X—C₁₋₅-alkylene-, wherein X is absent or wherein X is selected from C₃₋₆-cycloalkylene, 4- to 7-membered heterocyclyl, 5- to 10-membered heteroaryl and phenyl. In embodiments L¹ is independently at each occurrence —C₀₋₅-alkylene-X—C₁₋₅-alkylene-, wherein X is selected from C₃₋₆-cycloalkylene, 4- to 7-membered heterocyclyl, 5- to 10-membered heteroaryl and phenyl.

L¹ may be —X—C₁₋₅-alkylene-, e.g. —X—C₁₋₂-alkylene-. It may be that X is C₃₋₆-cycloalkylene, e.g. C₅-cycloalkylene. It may be that X is 5- to 10-membered heteroaryl, e.g. pyridinyl or pyrimidinyl. It may be that X is phenyl. It may be that L¹ is

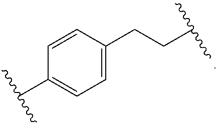 [SMILES: *CCc1ccc(*)cc1]

In the compound of Formula (I), and the salt of Formula (II) and (IV), it may be that R² and R³ are each independently selected from H, C₁₋₆-alkyl, C₃₋₆-cycloalkyl, 4- to 7-membered heterocyclyl, C₁₋₄-alkylene-R⁴, and C₂₋₄-alkylene-R⁵. In embodiments, R² and R³ are the same. Alternatively, R² and R³ are different.

In the compound of Formula (I), and the salt of Formula (II) and (IV), it may be that R² and R³ are both H. It may be that R² is H, and R³ is selected from C₁₋₆-alkyl, C₃₋₆-cycloalkyl, 4- to 7-membered heterocyclyl, C₁₋₄-alkylene-R⁴, and C₂₋₄alkylene-R⁵. It may be that neither R² nor R³ are H. Thus, it may be that R² and R³ are each independently selected from C₁₋₆-alkyl, C₃₋₆-cycloalkyl, 4- to 7-membered heterocyclyl, C₁₋₄-alkylene-R⁴, and C₂₋₄-alkylene-R⁵.

In the compound of Formula (I), and the salt of Formula (II) and (IV), it may be that R² and R³ are each independently selected from H, C₁₋₄-alkyl, C₃₋₆-cycloalkyl, 4- to 7-membered heterocyclyl, C₁₋₄-alkylene-R⁴, and C₂₋₄-alkylene-R⁵. It may be that R² and R³ are each independently selected from C₁₋₄-alkyl, C₃₋₆-cycloalkyl, 4- to 7-membered heterocyclyl, C₁₋₄-alkylene-R⁴, and C₂₋₄-alkylene-R⁵.

In the compound of Formula (I), and the salt of Formula (II) and (IV), it may be that R² and R³ are each independently selected from H and C₁₋₄-alkyl. Thus, it may be that R² and R³ are each independently selected from the group consisting of: H, methyl, ethyl, propyl, isopropyl, butyl, isobutyl, sec-butyl, and tert-butyl. It may be that R² is H, and R³ is selected from the group consisting of: methyl, ethyl, propyl, isopropyl, butyl, isobutyl, sec-butyl, and tert-butyl. It may be that R² is H and R³ is C₁₋₄-alkyl. It may be that R² is H and R³ is H. It may be that R² is C₁₋₄-alkyl and R³ is C₁₋₄-alkyl. It may be that R² is H and R³ is isopropyl. It may be that R² is methyl and R³ is methyl.

In the compound of Formula (I), and/or the salt of Formula (II) and (IV), it may be that L¹ and R² together with the nitrogen to which they are attached form a 3- to 6-membered heterocyclyl optionally substituted, where chemically possible, by 1 to 3 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein the heterocyclyl has 1 to 2 additional heteroatom ring vertices selected from the group consisting of O, N, and S. It may be that L¹ and R² together with the nitrogen to which they are attached form an unsubstituted 3- to 6-membered heterocyclyl. It may be that L¹ and R² together with the nitrogen to which they are attached form an unsubstituted 6-membered heterocyclyl.

In compound of Formula (I), and/or the salt of Formula (II) and (IV), R² and R³ together with the nitrogen to which they are attached may form a 3- to 6-membered heterocyclyl, e.g. morpholinyl.

In embodiments, R⁴ is selected from: C₃₋₆-cycloalkyl, 4- to 7-membered heterocyclyl, 5- to 10-membered heteroaryl and phenyl. Thus, in the compound of Formula (I), and the salt of Formula (II) and (IV), it may be that R² and R³ are each independently selected from H, C₁₋₄-alkyl, C₃₋₆-cycloalkyl, 4- to 7-membered heterocyclyl, C₁₋₄-alkylene-R⁴, and C₂₋₄-alkylene-R⁵, wherein R⁴ is selected from: C₃₋₆-cycloalkyl, 4- to 7-membered heterocyclyl, 5- to 10-membered heteroaryl and phenyl. It may be that R⁴ is phenyl. Thus, in the compound of Formula (I), and the salt of Formula (II) and (IV) it may be that R² and/or R³ is C₁₋₄-alkylene-phenyl. It may be that R² and/or R³ is benzyl.

In the carbamate of Formula (III), it may be that R³ is selected from H, C₁₋₆-alkyl, C₃₋₆-cycloalkyl, 4- to 7-membered heterocyclyl, C₁₋₄-alkylene-R⁴, and C₂₋₄-alkylene-R⁵. In embodiments, R³ is H. Alternatively, R³ is selected from C₁₋₆-alkyl, C₃₋₆-cycloalkyl, 4- to 7-membered heterocyclyl, C₁₋₄-alkylene-R⁴, and C₂₋₄-alkylene-R⁵. It may be that R³ is selected from H, C₁₋₄-alkyl, C₃₋₆-cycloalkyl, 4- to 7-membered heterocyclyl, C₁₋₄-alkylene-R⁴, and C₂₋₄-alkylene-R⁵.

In the carbamate of Formula (III), it may be that R³ is selected from H and C₁₋₄-alkyl. Thus, it may be that R³ is selected from the group consisting of: H, methyl, ethyl, propyl, isopropyl, butyl, isobutyl, sec-butyl, and tert-butyl. It may be that R³ is selected from the group consisting of: methyl, ethyl, propyl, isopropyl, butyl, isobutyl, sec-butyl, and tert-butyl. It may be that R³ is C₁₋₄-alkyl. It may be that R³ is isopropyl.

In embodiments, R⁴ is selected from: C₃₋₆-cycloalkyl, 4- to 7-membered heterocyclyl, 5- to 10-membered heteroaryl and phenyl. Thus, in the carbamate of Formula (III), it may be that R³ is selected from H, C₁₋₄-alkyl, C₃₋₆-cycloalkyl, 4- to 7-membered heterocyclyl, C₁₋₄-alkylene-R⁴, and C₂₋₄-alkylene-R⁵, wherein R⁴ is selected from: C₃₋₆-cycloalkyl, 4- to 7-membered heterocyclyl, 5- to 10-membered heteroaryl and phenyl. It may be that R⁴ is phenyl. Thus, in the carbamate of Formula (III), it may be that R³ is C₁₋₄-alkylene-phenyl.

In embodiments, R⁵ is selected from: OR⁶, CO₂R⁶, NR⁶R⁷, CONR⁶R⁷, SR⁶, and S(O)₂NR⁶R. It may be that R⁵ is selected from: OR⁶, NR⁶R⁷, and SR⁶.

In embodiments, R¹ is an azo dye, L¹ is —C₁₋₄-alkylene-, and R² and R³ are each independently selected from H, C₁₋₄-alkyl, C₃₋₆-cycloalkyl, 4- to 7-membered heterocyclyl, C₁₋₄-alkylene-R⁴, and C₂₋₄-alkylene-R⁵. It may be that R¹ is an azo dye, L¹ is —C₁₋₄-alkylene-, and R² and R³ are each H. It may be that R¹ is an azo dye, L¹ is —C₁₋₄-alkylene-, R² is H, and R³ is selected from C₁₋₄-alkyl, C₃₋₆-cycloalkyl, 4- to 7-membered heterocyclyl, C₁₋₄-alkylene-R⁴, and C₂₋₄-alkylene-R⁵. It may be that R¹ is an azo dye, L¹ is —C₁₋₄-alkylene-, and R² and R³ are each independently selected from C₁₋₄-alkyl, C₃₋₆-cycloalkyl, 4- to 7-membered heterocyclyl, C₁₋₄ alkylene-R⁴, and C₂₋₄-alkylene-R⁵.

In embodiments, R¹ is an azo dye, L¹ is —CH₂—, and R² and R³ are each independently selected from H, and C₁₋₄-alkyl. It may be that R¹ is an azo dye, L¹ is —CH₂—, and R² and R³ are each H. It may be that R¹ is an azo dye, L¹ is —CH₂—, R² is H, and R³ is C₁₋₄-alkyl. It may be that R¹ is an azo dye, L¹ is —CH₂—, and R² and R³ are each C₁₋₄-alkyl.

In embodiments, R¹ is an anthraquinone dye, L¹ is —C₁₋₄-alkylene-, and R² and R³ are each independently selected from H, C₁₋₄-alkyl, C₃₋₆-cycloalkyl, 4- to 7-membered heterocyclyl, C₁₋₄-alkylene-R⁴, and C₂₋₄-alkylene-R⁵. It may be that R¹ is an anthraquinone dye, L¹ is —C₁₋₄-alkylene-, and R² and R³ are each H. It may be that R¹ is an anthraquinone dye, L¹ is —C₁₋₄-alkylene-, R² is H, and R³ is selected from C₁₋₄-alkyl, C₃₋₆-cycloalkyl, 4- to 7-membered heterocyclyl, C₁₋₄-alkylene-R⁴, and C₂₋₄-alkylene-R⁵. It may be that R¹ is an anthraquinone dye, L¹ is —C₁₋₄-alkylene-, and R² and R³ are each independently selected from C₁₋₄-alkyl, C₃₋₆-cycloalkyl, 4- to 7-membered heterocyclyl, C₁₋₄-alkylene-R⁴, and C₂₋₄-alkylene-R⁵.

In embodiments, R¹ is an anthraquinone dye, L¹ is —CH₂—, and R² and R³ are each independently selected from H, and C₁₋₄-alkyl. It may be that R¹ is an anthraquinone dye, L¹ is —CH₂—, and R² and R³ are each H. It may be that R¹ is an anthraquinone dye, L¹ is —CH₂—, R² is H, and R³ is C₁₋₄-alkyl. It may be that R¹ is an anthraquinone dye, L¹ is —CH₂—, and R² and R³ are each C₁₋₄-alkyl.

In embodiments, it may be that the compound of Formula (I) is a compound of Formula (Ib):

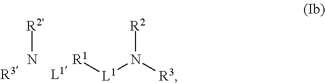

wherein:
R¹ is a dye;
L^{1′} has the same definition as L¹ as defined herein;
R² has the same definition as R² as defined herein; and
R^{3′} has the same definition as R³ as defined herein.

In this embodiment, it may be that R¹ is a disperse dye as defined herein. For example, it may be that R¹ is a disperse dye such as Disperse Blue 14.

In embodiments, it may be that the salt of Formula (II) is a salt of Formula (IIb):

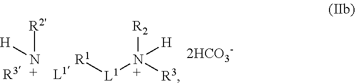

wherein:
R¹ is a dye;
L^{1′} has the same definition as L¹ as defined herein;
R^{2′} has the same definition as R² as defined herein; and
R^{3′} has the same definition as R³ as defined herein.

In this embodiment, it may be that R¹ is a disperse dye as defined herein. For example, it may be that R¹ is a disperse dye such as Disperse Blue 14.

In embodiments, it may be that the carbamate of Formula (III) is a carbamate of Formula (IIIb):

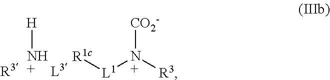

wherein:
R¹ is a dye;
L^{1′} has the same definition as L¹ as defined herein; and R^{3′} has the same definition as R³ as defined herein.

In this embodiment, it may be that R¹ is a disperse dye as defined herein. For example, it may be that R¹ is a disperse dye such as Disperse Blue 14.

In embodiments, it may be that L¹ and L^{1′} are the same. It may be that L¹ and L^{1′} are different. In embodiments, it may be that R² and R^{2′} are the same. It may be that R² and R^{2′} are different. In embodiments, it may be that R³ and R^{3′} are the same. It may be that R³ and R^{3′} are different.

In embodiments, it may be that the salt of Formula (IV) is a salt of Formula (IVb):

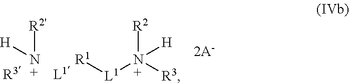

wherein:
R¹ is a dye;
L^{1′} has the same definition as L¹ as defined herein;
R^{2′} has the same definition as R² as defined herein; and
R^{3′} has the same definition as R³ as defined herein.

In embodiments, R¹ is an anthraquinone dye, L¹ is —CH₂—, and R² and R³ are each independently.

In this embodiment, it may be that R¹ is a disperse dye as defined herein. For example, it may be that R¹ is a disperse dye such as Disperse Blue 14.

In the salt of Formula (IV), it may be that A¹ is a halide (e.g. chloride, bromide, fluoride or iodide); a hydrogen sulfate; a C₁-C₆-alkyl sulfate (e.g. methylsulfate or ethylsulfate); a carboxylate (e.g. formate, acetate, citrate, tartrate or oxalate); a C₁-C₆-alkylsulfonate (e.g. mesylate); or an arylsulfonate, wherein the aryl, preferably phenyl, is optionally substituted with one to four C₁-C₄ alkyl groups (e.g. p-toluenesulfonate). A¹ may be a halide. A¹ may be chloride.

It may be the salt of Formula (II) is in a form that is substantially free of any compound of Formula (I). It may be that the salt of Formula (II) is in a form that comprises no more than 75 mol % of the compound Formula (I), e.g. no more than 50 mol %, or no more than 25 mol %, of the compound of Formula (I). It may be that the salt of Formula (II) is in a form that comprises no more than 10 mol % of the compound Formula (I), e.g. no more than 5 mol %, or no more than 2.5 mol %, of the compound of Formula (I).

It may be the carbamate of Formula (III) is in a form that is substantially free of any compound of Formula (I). It may be that the carbamate of Formula (III) is in a form that comprises no more than 75 mol % of the compound Formula (I), e.g. no more than 50 mol %, or no more than 25 mol %, of the compound of Formula (I). It may be that the carbamate of Formula (III) is in a form that comprises no more than 10 mol % of the compound Formula (I), e.g. no more than 5 mol %, or no more than 2.5 mol %, of the compound of Formula (I).

### The Dye(s)

In embodiments, R¹ is a dye. Suitable dyes include disperse dyes, solvent dyes, vat dyes, sulphur dyes, mordant dyes, acid dyes, direct dyes and reactive dyes. The disperse dyes, solvent dyes, vat dyes, sulphur dyes, mordant dyes, acid dyes, direct dyes and reactive dyes that can be used in the compounds and methods of the invention include all dyes classified as such in The Colour Index™ published by the Society of Dyers and Colourists (SDC) and American Association of Textile Chemists and Colourists (AATCC). In certain embodiments, the disperse dyes, solvent dyes, vat dyes, sulphur dyes, mordant dyes, acid dyes, direct dyes and reactive dyes that can be used in the compounds and methods of the invention may include all dyes classified as such in The Colour Index™ on the 1 May 2017.

Typically, the dye absorbs light within the visible spectrum (i.e. wavelengths ranging from approximately 400 to 700 nanometers). It may be that the dye absorbs light within the ultraviolet spectrum (i.e. wavelengths ranging from approximately 10 to 400 nanometers).

In preferred embodiments, R¹ is a disperse dye. The disperse dye may be selected from the group consisting of azo, anthraquinone, nitro, aminoketone, methine, or naphthoquinone dyes. It may be that R¹ is an azo dye. It may be that R¹ is an anthraquinone dye.

In other embodiments, it may be that R¹ is not a disperse dye.

The term “dye” is used in this specification in the context of the compounds of the invention to mean a dye molecule (e.g. a disperse dye), wherein at least one hydrogen, halogen, C₁-C₄-alkyl (e.g. methyl), C₁-C₄-carboxylate (e.g. acetate) or a nucleophilic functional group (e.g., —OH, —NH₂) is absent and the atom to which it was attached connects the rest of the dye molecule with the rest of the compound of formula (I), (II), (III) or (IV). The “dye” may be a compound comprising a chromophore.

As defined herein, R¹ may be a “disperse dye”. The term “disperse dye” is used in this specification in the context of the compounds of the invention to mean a disperse dye molecule, wherein at least one hydrogen, C₁-C₄-alkyl (e.g. methyl), C₁-C₄-carboxylate (e.g. acetate) or a nucleophilic functional group (e.g., —OH, —NH₂) is absent and the atom to which it was attached connects the rest of the disperse dye molecule with the rest of the compound of formula (I), (II), (III), or (IV).

For example, Disperse Red 1 (DR1) is a common azo dye with the following structure:

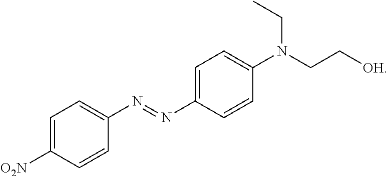

In the context of the present invention, it may be that R¹ is DR1, wherein an —OH is removed from DR1, such that R¹ has the following structure:

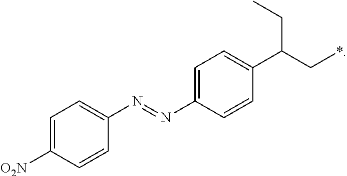

wherein * is the point of attachment to the remainder of Formula (I)—(IV). Thus, it may be that Formula (I) has the following structure, wherein R¹ is DR1:

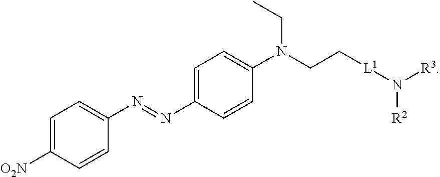

In another example, Disperse Blue 22 (DB22) is a common anthraquinone dye with the following structure:

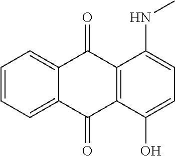 [SMILES: CNc1ccc(O)c2c1C(=O)c1ccccc1C2=O]

In the context of the present invention, it may be that R¹ is DB22, wherein a methyl group is omitted from DB22, such that R¹ has the following structure:

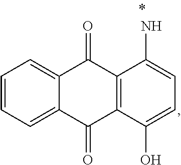

wherein * is the point of attachment to the remainder of Formula (I)—(IV). Thus, it may be that Formula (I) has the following structure, wherein R¹ is DB22:

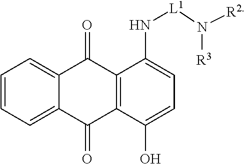

In a further example, Disperse Blue 14 (DB14) is a common anthraquinone dye with the following structure:

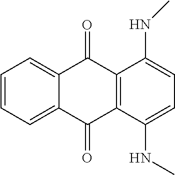 [SMILES: CNc1ccc(NC)c2c1C(=O)c1ccccc1C2=O]

In the context of the present invention, it may be that R¹ is DB14, wherein two methyl groups are omitted from DB14, such that R¹ has the following structure:

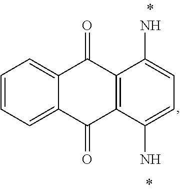

wherein * is the point of attachment to the remainder of Formula (Ib)-(IVb). Thus, it may be that Formula (Ib) has the following structure, wherein R¹ is DB14:

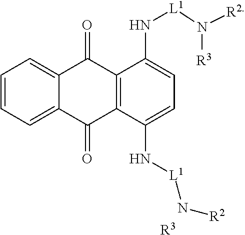

In a further example, Disperse Blue 60 (DB60) is a common anthraquinone dye with the following structure:

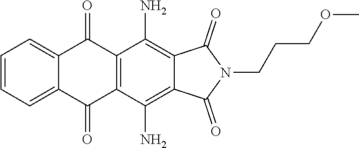 [SMILES: COCCCn1c(=O)c2c(N)c3c(=O)c4ccccc4c(=O)c3c(N)c2c1=O]

In the context of the present invention, it may be that R¹ is DB60, wherein the methoxypropyl groups is omitted from DB60, such that R¹ has the following structure:

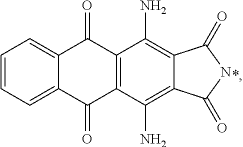

wherein * is the point of attachment to the remainder of Formula (I)—(IV). Therefore, it may be that Formula (I) has the following structure, wherein R¹ is DB14:

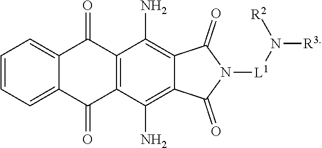

In another example, Disperse Yellow 3 (DY3) is a common azo dye with the following structure:

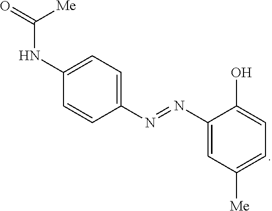 [SMILES: CC(=O)Nc1ccc(/N=N/c2cc(C)ccc2O)cc1]

In the context of the present invention, it may be that R¹ is DY3, wherein an acetate group is omitted from DY3, such that R¹ has the following structure:

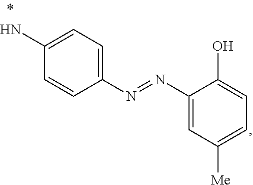

wherein * is the point of attachment to the remainder of Formula (I). Thus, it may be that Formula (I) has the following structure, wherein R¹ is DY3:

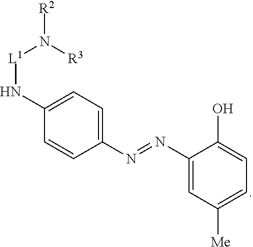

In another example, Disperse Yellow 26 (DY26) is a nitrodiphenylamine dye with the following structure:

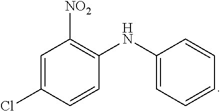 [SMILES: O=[N+]([O-])c1cc(Cl)ccc1Nc1ccccc1]

In the context of the present invention, it may be that R¹ is DY26, wherein a Cl group is omitted from DY26, such that R¹ has the following structure:

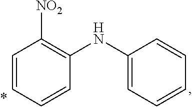

wherein * is the point of attachment to the remainder of Formula (I)—(IV). Thus, it may be that Formula (I) has the following structure, wherein R¹ is DY26:

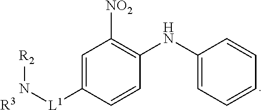

The compounds of the present invention may be a different colour to the (parent) dye.

### The Method of Dyeing Fibres

In embodiments, the dye liquor of step a) comprises water, and a salt of Formula (II), or a carbamate of Formula (III), or mixtures thereof and the dye liquor of step a) is obtained by:
i) obtaining an aqueous dye liquor comprising water and a compound of Formula (I) as described herein; and
ii) exposing the aqueous dye liquor to carbon dioxide to form the dye liquor.

In embodiments, in step i), the aqueous dye liquor comprises water and a compound of Formula (I), wherein R¹ is a disperse dye. Accordingly, in this embodiment, in step a), the dye liquor comprises water and a salt of Formula (II) or a carbamate of Formula (III), wherein R¹ is a disperse dye. Preferably, R¹ is an azo dye or an anthraquinone dye.

In embodiments, the dye liquor comprises water, and a salt of Formula (II). The dye liquor of step a) may be obtained by dissolving the salt of Formula (II) in water. The salt of Formula (II) may be obtained according to the eighth aspect of the invention.

In other embodiments, the dye liquor comprises water, and a carbamate of Formula (III). Alternatively, it may be that the dye liquor comprises a mixture of both a salt of Formula (II) and a carbamate of Formula (III).

In embodiments, the dye liquor may comprise at least one salt of Formula (II) and/or carbamate of Formula (III). It may be that the dye liquor comprises one salt of Formula (II) and/or carbamate of Formula (III). It may be that the dye liquor comprises more than one salt of Formula (II) and/or carbamate of Formula (III), for example, two different salts of Formula (II).

In embodiments, the aqueous dye liquor may comprise at least one compound of Formula (I). It may be that the aqueous dye liquor comprises one compound of Formula (I).

It may be that the aqueous dye liquor comprises more than one compound of Formula (I), for example, two different compounds of Formula (I).

In embodiments, the dye liquor of step a) comprises water, a compound of Formula (I), and carbon dioxide. The dye liquor should contain CO₂ at a concentration higher than the natural concentration of CO₂ in water (at the temperature and atmosphere used). Typically, the carbon dioxide concentration in the dye liquor will be at least 3 mM. It may be that the carbon dioxide concentration is at least 5 mM, e.g. at least 10 mM. It may be that the carbon dioxide concentration is at least 20 mM, e.g. at least 30 mM. It may be that the carbon dioxide concentration is at least 40 mM, e.g. at least 50 mM. It may be that the carbon dioxide concentration is at least 60 mM, e.g. at least 70 mM. It may be that the dye liquor is saturated with CO₂.

It may be that the carbon dioxide concentration in the dye liquor is at least 50 mM, e.g. at least 60, 70 or 80 mM, the weight ratio of the dye liquor to the fibres (i.e. the dye liquor ratio) is from about 5:1 to 15:1 and the amount of the compound of the invention used in the dye liquor is in the range from 0.1% to 10% of the mass of the fibres (e.g. 0.1% to 10% on mass of fibre).

In embodiments, the dye liquor of step a) comprises a salt of Formula (IV) according to the sixth aspect of the invention and a metal bicarbonate salt.

Typically, the salt of Formula (IV) and the metal bicarbonate salt may be in stoichiometric amounts. The salt of Formula (IV) and the metal bicarbonate salt may be in a molar ratio of from 1:2 to 2:1. The salt of Formula (IV) and the metal bicarbonate salt may be in a molar ratio of from 2:3 to 3:2. The salt of Formula (IV) and the metal bicarbonate salt may be in a molar ratio of from 1:1.2 to 1.2:1. The metal bicarbonate salt may be sodium bicarbonate or potassium bicarbonate.

In embodiments, the aqueous dye liquor and/or dye liquor do not comprise a dispersing agent. Typical dispersing agents include anionic, polyelectrolyte, compounds (and mixtures thereof), such as lignin sulfonates or formaldehyde polycondensates of arylsulfonic acids (e.g. disodium methylenebisnaphthalene sulfonate, sodium oleyl-p-anisidinesulfonate). Thus, it may be that the aqueous dye liquor does not comprise a dispersing agent. It may be that the dye liquor does not comprise a dispersing agent. Preferably, it may be that the aqueous dye liquor and the dye liquor do not comprise a dispersing agent.

In embodiments, it may be that the aqueous dye liquor and/or dye liquor do not comprise a carrier. Typical carriers include, for example, o-dichlorobenzene, 1,2,4-trichlorobenzene, dimethyl phthalate, diallyl phthalate, o-phenyl phenol, p-phenyl phenol, diphenyl, 1-methylnaphthalene, ethylene carbonate and propylene carbonate. Thus, it may be that the aqueous dye liquor does not comprise a carrier. It may be that the dye liquor does not comprise a carrier. Preferably, it may be that the aqueous dye liquor and the dye liquor do not comprise a carrier. The inventors have found that excellent colour strength can be obtained using the method of the invention in the absence of a dispersing agent or a carrier.

Alternatively, the aqueous dye liquor and/or dye liquor may comprise at least one additive selected from: a dispersing agent, a carrier, a stabiliser, a surfactant, an antioxidant, a pH modifier/buffer, lubricant, softener, hydrotrope, wetting agent and migrating agent. The aqueous dye liquor and/or dye liquor may comprise at least one additive selected from: a stabiliser, a surfactant, an antioxidant, a pH modifier/buffer, lubricant, softener, hydrotrope, wetting agent and migrating agent. The aqueous dye liquor and/or dye liquor may comprise a carrier. The aqueous dye liquor and/or dye liquor may comprise a UV absorber (e.g. a benzophenone or a triazine).

Suitably, the fibres comprise any of the fibres described herein. It may be that the fibres are polyester fibres. Preferably, the fibres are poly(ethylene terephthalate) fibres.

It may be that when the fibres are polyester fibres, e.g. poly(ethylene terephthalate) fibres, in step i), the aqueous dye liquor comprises water and a compound of Formula (I), wherein R¹ is a disperse dye. Thus, it may be that the polyester fibres, e.g. poly(ethylene terephthalate) fibres are dyed with a compound of Formula (I), wherein R¹ is a disperse dye. Preferably, R¹ is an azo dye or an anthraquinone dye. In embodiments, in step b), it may be that the fibres wetted with the dye liquor are heated to a temperature of from about 90° C. to 180° C., 100° C. to 160° C., or 120° C. to 140° C. Preferably, it may be that the fibres wetted with the dye liquor are heated to a temperature of from about 120° C. to 140° C. More preferably, it may be that the fibres wetted with the dye liquor are heated to a temperature of about 130° C.

In embodiments, in step b), it may be that the fibres wetted with the dye liquor are heated to temperature for a period of from about 5 min to 120 min, 20 min to 100 min, or 40 min to 80 min. Preferably, it may be that the fibres wetted with the dye liquor are heated to temperature for a period of about 60 min. Thus, it may be that the fibres wetted with the dye liquor are heated to about 130° C. for a period of about 60 min.

In embodiments, in step b), it may be that the fibres wetted with the dye liquor are heated to temperature, at a temperature ramp of from about 0.5° C./min to 5° C./min, 1° C./min to 3° C./min, or 1.5° C./min to 2° C./min. Preferably, it may be that the fibres wetted with the dye liquor are heated to temperature, at a temperature ramp of from about 1.5° C./min to 2° C./min.

Thus, it may be that in step b), it may be that the fibres wetted with the dye liquor are heated to about 90° C. to 180° C. at a temperature ramp of from about 0.5° C./min to 5° C./min. Preferably, it may be that the fibres wetted with the dye liquor are heated to about 120° C. to 150° C. at a temperature ramp of from about 1.5° C./min to 2° C./min.

The method may be conducted at a pressure of about 1 atm. The method may be conducted at a pressure in the range from 0.9 atm to 3 atm. The method may be conducted at an elevated pressure, e.g. a pressure greater than 0.9 atm and up to 5 atm.

The carbon dioxide may be in the form of a gas. If a gas, it is typically bubbled through the dye liquor. The carbon dioxide may be in solid form, e.g. pellets. In embodiments, in step ii), the carbon dioxide is bubbled through the aqueous dye liquor comprising water and at least one compound of Formula (I) as described herein. It may be that the aqueous dye liquor is saturated with CO₂.

It may be that the weight ratio of the dye liquor to the fibres (i.e. the dye liquor ratio) is from about from 5:1 to 100:1, 5:1 to 50:1, or 5:1 to 15:1. Preferably, it may be that the weight ratio of the dye liquor to the fibres (i.e. the dye liquor ratio) is about 10:1. For the avoidance of doubt, it may be that 1 g of fibres is dyed in 10 mL of dye liquor.

It may be that the amount of the compound of the invention used in the dye liquor is in the range from 0.1% to 10% of the mass of the fibres (e.g. 0.1% to 10% on mass of fibre). It may be that the amount of the compound of the invention used in the dye liquor is in the range from 0.5% to 10% of the mass of the fibres (e.g. 0.5% to 10% on mass of fibre). It may be that the amount of the compound used in the dye liquor is in the range from 1% to 5% of the mass of the fibres (e.g. 1% to 5% on mass of fibre). It may be that the amount of the compound used in the dye liquor is 0.5%, 1%, 1.5%, 2%, 2.5%, 3%, 3.5%, 4%, 4.5%, 5%, 5.5%, 6%, 6.5%, 7%, 7.5%, 8%, 8.5%, 9%. 9.5%, or 10% of the mass of the fibres.

When the aqueous dye liquor comprising water and a compound of Formula (I) is exposed to carbon dioxide (in step ii)), it forms a dye liquor comprising water and a salt of Formula (II) and/or carbamate of Formula (III). Thus, the compound of Formula (I) is converted to the salt of Formula (II) and/or carbamate of Formula (III) upon exposure to carbon dioxide in an aqueous solution. It may be that the dye liquor is a homogenous solution of the salt of Formula (II). It may be that the dye liquor is a homogenous solution of the carbamate of Formula (III).

It may be that the dye liquor of step a) comprises a salt of Formula (II), a carbamate of Formula (III), or mixtures thereof, and substantially no compound of Formula (I). It may be that, if present, the compound Formula (I) makes up less than 75 mol %, e.g. less than 50 mol %, or less than 25 mol % of the combined moles of the salt of Formula (II), the carbamate of Formula (III) and the compound of Formula (I) in the dye liquor. It may be that, if present, the compound Formula (I) makes up less than 10 mol %, e.g. less than 5 mol %, or less than 2.5 mol %, of the combined moles of the salt of Formula (II), the carbamate of Formula (III) and the compound of Formula (I) in the dye liquor.

When the dye liquor is exposed to elevated temperatures of from 90° C. to 180° C. (in step b)), the salt of Formula (II) and/or carbamate of Formula (III) is converted back to the compound of Formula (I). Thus, the compound of Formula (I) is ejected from the dye liquor to penetrate and dye the fibres. As such, the exhausted dye liquor comprises water and any remaining compound of Formula (I), salt of Formula (II) and/or carbamate of Formula (III).

In embodiments, step c) comprises reducing the temperature of the exhausted dye liquor prior to removal of the dyed fibres from the exhausted dye liquor. Thus, it may be that the exhausted dye liquor is cooled to a temperature of about 20° C. to about 80° C. It may be the exhausted dye liquor is cooled to a temperature of about 30° C. to about 60° C. It may be the exhausted dye liquor is cooled to a temperature of about 35° C. to about 45° C. Preferably, it may be that the exhausted dye liquor is cooled to a temperature of about 40° C. before the dyed fibres are removed from the exhausted dye liquor.

In embodiments, the exhausted dye liquor is cooled to a temperature as described herein, and held at said temperature for about 1 min to about 30 min, about 5 min to about 20 min, or about 5 min to about 15 min. Preferably, it may be that the exhausted dye liquor is cooled to a temperature as described herein, and held at said temperature for about 10 min. Thus, it may be that the exhausted dye liquor is cooled to a temperature of about 40° C. and held at said temperature for about 10 min, before the dyed fibres are removed from the exhausted dye liquor.

In embodiments, the dye liquor in step a) can be reformed from the exhausted dye liquor in step c), upon addition of a further compound of Formula (I) and exposure to carbon dioxide. Accordingly, the method may further comprise steps d and e):
d) subjecting the exhausted dye liquor to a compound of Formula (I) to form a recycled aqueous dye liquor; and
e) exposing the recycled aqueous dye liquor to carbon dioxide to form the dye liquor.
   Thus, it may be that the method comprises:
a) subjecting the fibres to a dye liquor comprising water, and a salt of Formula (II) as described herein, or a carbamate of Formula (III) as described herein, or mixtures thereof, to provide the fibres wetted with the dye liquor;
b) heating the fibres wetted with the dye liquor to a temperature of from 90° C. to 180° C., to provide dyed fibres wetted with an exhausted dye liquor comprising water;
c) removing the dyed fibres from the exhausted dye liquor;
d) subjecting the exhausted dye liquor to a compound of Formula (I) to form a recycled aqueous dye liquor; and
e) exposing the recycled aqueous dye liquor to carbon dioxide to reform the dye liquor of step a).

It may be that the dye liquor is recycled once. Thus, it may be that the method comprises performing the following steps, in order: a), b), c), d), e), a), b), and c). It may be that the dye liquor is recycled more than once. Thus, it may be that the dye liquor is recycled up to ten times. It may be that the dye liquor is recycled up to five times. Thus, it may be that the dye liquor is recycled indefinitely. It may be that the dye liquor is subjected to one or more purification steps to remove undesired by products before being reused in step a).

The skilled person will be aware that when the dye liquor is recycled, the first time step a) is performed, it may be that the dye liquor in step a) is obtained by steps i) and ii). For the avoidance of doubt, it may be that the dye liquor is recycled once, and thus the method may comprise performing the following steps, in order: i), ii), a), b), c), d), e), a), b), and c).

When the dye liquor is recycled, it may be that the exhausted dye liquor (in step c)) is cooled to a temperature of about 50° C. to about 100° C. It may be that the exhausted dye liquor (in step c)) is cooled to a temperature of about 60° C. to about 90° C. It may be that the exhausted dye liquor (in step c)) is cooled to a temperature of about 70° C. to about 80° C.

The method may be followed by reduction clearing, acid clearing, rinsing and/or optionally further treatments depending on the nature of the substrate (e.g. whether the substrate is a single or multicomponent blend of fibres), and end-use requirement. Exemplary further treatments include softening, heat setting, etc.

### The Method of Acid Clearing

Due to the terminal amine present in the compounds of the invention, they can be protonated under mildly acidic conditions. The protonated dye can then be washed from the dyebath using an aqueous solution. Due to the lack of permeability below the boiling point of water, this process only removes surface dye without effecting the dye that has been absorbed into the fibre. This means that the compounds of the invention can be cleared using a novel acidic clearing method, which negates the need for reductive clearing.

Thus, also provided is a method of acid clearing, wherein the fibres are dyed with a compound of Formula (I) as described herein, the method comprising the following steps:
a) subjecting the dyed fibres to an acidic solution, and heating the dyed fibres wetted with the acidic solution; and
b) removing the dyed fibres from the acidic solution.

In embodiments, the method further comprises rinsing and drying the removed dyed fibres. It may be that the rinsing step removes the protonated dye (i.e. a salt of Formula (II) and/or a carbamate of Formula (III)) from the surface of the fibres.

In embodiments, the fibres are dyed with a compound of Formula (I), wherein R¹ is a dye selected from the group consisting of: disperse dyes, solvent dyes, vat dyes, sulphur dyes, mordant dyes, acid dyes, direct dyes and reactive dyes. It may be that the fibres are dyed with a compound of Formula (I), wherein R¹ is a dye selected from the group consisting of: solvent dyes, vat dyes, sulphur dyes, mordant dyes, acid dyes, direct dyes and reactive dyes (i.e. R¹ is not a disperse dye).

In other embodiments, the fibres are dyed with a compound of Formula (I), wherein R¹ is a disperse dye. The disperse dye may be selected from the group consisting of azo, anthraquinone, nitro, aminoketone, methine, or naphthoquinone dyes.

In embodiments, the fibres have been dyed with one or more different dye compounds. Thus, it may be that the fibres have been dyed with one dye compound (e.g. a compound of Formula (I), wherein R¹ is a disperse dye). It may be that the fibres have been dyed with two different dye compounds (e.g. a compound of Formula (I) wherein R¹ is a disperse dye, and a compound of Formula (I) wherein R¹ is not a disperse dye). It may be that the fibres have been dyed with three different dye compounds.

In embodiments, the fibres have been dyed by a method according to the fourth aspect of the invention.

Suitably, the fibres comprise any of the fibres described herein. It may be that the fibres are polyester fibres. Preferably, the fibres are poly(ethylene terephthalate) fibres.

In embodiments, the acidic solution comprises water and an acid selected from formic acid, acetic acid, and citric acid. It may be that the acidic solution is an aqueous solution comprising from about 0.1 wt. % to 5 wt. % acid, 0.1 wt. % to 3 wt. % acid, or 0.5 wt/% to 2 wt. % acid. For example, it may be that the acidic solution is an aqueous solution comprising 2 wt. % formic acid.

In embodiments, the dyed fibres wetted with the acidic solution are heated to a temperature of less than about 100° C. It may be that the dyed fibres wetted with the acidic solution are heated to a temperature of from about 30° C. to 100° C., 50° C. to 90° C., or 60° C. to 80° C. Preferably, it may be that the dyed fibres wetted with the acidic solution are heated to a temperature of about 70° C.

In embodiments, the dyed fibres wetted with the acidic solution are heated to a temperature for a period of from about 5 min to 120 min, 20 min to 100 min, or 40 min to 80 min. Preferably, it may be that the dyed fibres wetted with the acidic solution are heated to temperature for a period of about 60 min. Thus, it may be that the dyed fibres wetted with the acidic solution are heated to about 70° C. for a period of about 60 min.

When the dyed fibres are wetted with the acidic solution, the excess dye at the surface of the fibres in the form of the compound of Formula (I) is converted to the salt of Formula (II). The salt of Formula (II) is ejected from the surface of the dyed fibres and diffuse into the acidic solution. Thus, providing dyed fibres wetted with the acidic solution, wherein the acidic solution further comprises the salt of Formula (II).

In embodiments, step b) comprises reducing the temperature of the acidic solution prior to removal of the dyed fibres from the acidic solution. Thus, it may be that the acidic solution is cooled to a temperature of about 20° C. to about 80° C. It may be the acidic solution is cooled to a temperature of about 30° C. to about 60° C. It may be the acidic solution is cooled to a temperature of about 35° C. to about 45° C. Preferably, it may be that the acidic solution is cooled to a temperature of about 40° C. before the dyed fibres are removed from the acidic solution.

As discussed above, the method may be followed by rinsing and drying, and optionally further treatments depending on the nature of the substrate (e.g. whether the substrate is a single or multicomponent blend of fibres), and end-use requirement. Exemplary further treatments include softening, heat setting, etc.

The Method of Decolouring Dyed Fibres with Carbon Dioxide

In a fifth aspect of the invention, there is provided a method of decolouring dyed fibres, wherein the fibres are dyed with a compound of Formula (I) as described herein, the method comprising exposing the dyed fibres to a liquid comprising carbon dioxide to provide decoloured fibres.

The liquid may be an aqueous liquid. Thus, the method may comprise the following steps:
a) wetting the dyed fibres with an aqueous liquid;
b) exposing the dyed fibres wetted with the aqueous liquid to carbon dioxide to provide decoloured fibres wetted with a decolouration liquor, said decolouration liquor comprising water, a salt of Formula (II) as described herein, and/or a carbamate of Formula (III) as described herein, or mixtures thereof; and
c) removing the decoloured fibres from the decolouration liquor.

The aqueous liquid may be water. The aqueous liquid may be an aqueous solution.

The liquid may be an alcoholic liquid. The method may comprise the following steps:
a) wetting the dyed fibres with an alcoholic liquid;
b) exposing the dyed fibres wetted with the alcoholic solution to carbon dioxide to provide decoloured fibres wetted with a decolouration liquor, said decolouration liquor comprising alcohol, a salt of Formula (II) as described herein, and/or a carbamate of Formula (III) as described herein, or mixtures thereof; and
c) removing the decoloured fibres from the decolouration liquor.
The alcoholic liquid may be a C₁-C₄-alcohol, e.g. ethanol, methanol, isopropanol. The alcoholic liquid may be an alcoholic solution.

The liquid may be liquid CO₂. The method may comprise the following steps:
d) wetting the dyed fibres with liquid CO₂ to provide decoloured fibres wetted with a decoloration liquor, said decolorisation liquor comprising CO₂ a carbamate of Formula (III) as described herein; and
e) removing the decoloured fibres from the decoloration liquor.

In embodiments, the fibres are dyed with a compound of Formula (I), wherein R¹ is a dye selected from the group consisting of: disperse dyes, solvent dyes, vat dyes, sulphur dyes, mordant dyes, acid dyes, direct dyes and reactive dyes. It may be that the fibres are dyed with a compound of Formula (I), wherein R¹ is a dye selected from the group consisting of: solvent dyes, vat dyes, sulphur dyes, mordant dyes, acid dyes, direct dyes and reactive dyes (i.e. R¹ is not a disperse dye).

In other embodiments, the fibres are dyed with a compound of Formula (I), wherein R¹ is a disperse dye. The disperse dye may be selected from the group consisting of azo, anthraquinone, nitro, aminoketone, methine, or naphthoquinone dyes.

In embodiments, the fibres have been dyed with one or more different dye compounds. Thus, it may be that the fibres have been dyed with one dye compound (e.g. a compound of Formula (I), wherein R¹ is a disperse dye). It may be that the fibres have been dyed with two different dye compounds (e.g. a compound of Formula (I) wherein R¹ is a disperse dye, and a compound of Formula (I) wherein R¹ is not a disperse dye). It may be that the fibres have been dyed with three different dye compounds.

In embodiments, the fibres have been dyed by a method according to the fourth aspect of the invention. In embodiments, it may be that the fibres have also been subjected to a method of acid clearing, as described herein.

Suitably, the fibres comprise any of the fibres described herein. It may be that the fibres are polyester fibres. Preferably, the fibres are poly(ethylene terephthalate) fibres.

When the dyed fibres wetted with the liquid are exposed to carbon dioxide, the compound of Formula (I) is converted to the salt of Formula (II) and/or the carbamate of Formula (III). The salt of Formula (II) and/or the carbamate of Formula (III) are ejected from the dyed fibres and diffuse into the liquid to form a decolorisation liquor. Thus, providing decoloured fibres wetted with the decolorisation liquor. Where an aqueous liquid is used, the decolorisation liquor comprises water, the salt of Formula (II) and/or the carbamate of Formula (III).

In embodiments, at least 50% of the dye compound is ejected from the dyed fibres, e.g. at least 75% of the dye compound, 90% of the dye compound, 95% of the dye compound, or at least 99% of the dye compound is ejected from the dyed fibres. This can be determined by measuring reflectance using a spectrophotometer. From this K/S can be derived which is proportional to concentration of the dye compound. Alternatively, UV-Vis of the extracted liquor can also provide concentration values. The ejection can be calculated from these concentration values and knowledge of the starting concentration.

In embodiments, in step b), the dyed fibres wetted with the liquid may be exposed to carbon dioxide at a pressure of about 30 bar to about 50 bar.

In embodiments, in step b), the dyed fibres wetted with the liquid may be exposed to carbon dioxide at a temperature of about 90° C. to about 150° C.

In embodiments, the decolorisation liquor in step c) or step e) may be recycled and used in a method of dyeing fibres as described herein.

In embodiments, the decoloured fibres removed from the decolorisation liquor in step c) may be recycled and used in a method of dyeing fibres as described herein.

It may be that steps a) to c) are repeated more than once. It may be that steps a) to c) are repeated twice. For example, it may be that the decoloured fibres in step c) are subjected to steps a) to c), such that the method comprises performing the following steps, in order: a), b), c), a), b), and c). It may be that steps a) to c) are repeated up to ten times. It may be that steps a) to c) are repeated up to five times. It may be that steps d) and e) are repeated more than once. It may be that steps d) and e) are repeated twice. For example, it may be that the decoloured fibres in step e) are subjected to steps d) and e), such that the method comprises performing the following steps, in order: d), e), d), and e). It may be that steps d) and e) are repeated up to ten times. It may be that steps d) and e) are repeated up to five times.

The method may be followed by rinsing, drying, and optionally further treatments depending on the nature of the substrate (e.g. whether the substrate is a single or multicomponent blend of fibres), and end-use requirement. Exemplary further treatments include softening, heat setting, etc.

### The Fibres

The fibres may be present as individual fibres. The fibres may be present as a fibre substrate, for example, as a fabric, a garment, or part of a garment.

The fibres may comprise synthetic fibres or natural fibres or a mixture thereof. The fibres may comprise synthetic fibres. The fibres may comprise fibres selected from: a polyester, a polyamide, a polyurethane, a polyalkylene, a polyacrylonitrile, natural or regenerated protein (e.g. wool, silk), natural or regenerated cellulose, cellulose ester, hair, polyvinyl chloride, carbon or a mixture thereof.

Exemplary polyesters include poly(ethylene terephthalate) (PET), poly(butylene terephthalate) (PBT), poly(lactic acid) (PLA), polycaprolactone, polyesters based on furan dicarboxylic acid, and poly(trimethylene terephthalate) (PTT). Exemplary polyurethanes include Lycra®. Exemplary polyamides include nylon.

The fibres may be or may comprise polyamide fibres, e.g. nylon fibres.

The fibres may comprise polyester fibres or a mixture of a polyester with a fibre selected from cotton, wool, silk and polyurethane (e.g. Lycra®). The fibres may comprise PET fibres or a mixture of PET with a fibre selected from cotton, wool, silk and polyurethane (e.g. Lycra®). The fibres may be polyester fibres. The fibres may be poly(ethylene terephthalate) (PET) fibres. The fibres may comprise a mixture of a polyester with a fibre selected from cotton, wool, silk and polyurethane (e.g. Lycra®). The fibres may comprise a mixture of PET with a fibre selected from cotton, wool, silk and polyurethane (e.g. Lycra®).

The fibres may comprise silk or wool fibres.

Preferably, the fibres are polyester fibres. More preferably, the fibres are poly(ethylene terephthalate) fibres.

### Method of Forming a Salt of Formula (II)

In the seventh aspect of the invention, there is provided a method of forming a salt of Formula (II) according to the second aspect of the invention, a carbamate of Formula (III) according to the third aspect of the invention, or a mixture thereof, the method comprising contacting a compound of Formula (I) according to the first aspect of the invention with carbon dioxide.

The method may comprise exposing a solution comprising the compound of Formula (I) to carbon dioxide. The solution may be an aqueous solution.

The method may be conducted at a pressure of about 1 atm. The method may be conducted at a pressure in the range from 0.9 atm to 3 atm. The method may be conducted at an elevated pressure, e.g. a pressure greater than 0.9 atm and up to 5 atm.

The carbon dioxide may be in the form of a gas. If a gas, it is typically bubbled through a solution (e.g. an aqueous solution) comprising the compound of Formula (I). The carbon dioxide may be in solid form, e.g. pellets. The solution should contain CO₂ at a concentration higher than the natural concentration of CO₂ in water (at the temperature and atmosphere used). Typically, the carbon dioxide concentration in the solution will be at least 3 mM. It may be that the carbon dioxide concentration is at least 5 mM, e.g. at least 10 mM. It may be that the carbon dioxide concentration is at least 20 mM, e.g. at least 30 mM. It may be that the carbon dioxide concentration is at least 40 mM, e.g. at least 50 mM. It may be that the carbon dioxide concentration is at least 60 mM, e.g. at least 70 mM. It may be that the solution is saturated with CO₂.

In the eighth aspect of the invention there is provided a method of forming a salt of Formula (II) according to the second aspect of the invention, the method comprising contacting a salt of Formula (IV) according to the sixth aspect of the invention with a metal bicarbonate salt.

The method may comprise dissolving both the salt of Formula (IV) and the metal bicarbonate salt in an organic solvent, e.g. methanol. Typically, the salt of Formula (IV) and the metal bicarbonate salt are in stoichiometric amounts. The salt of Formula (IV) and the metal bicarbonate salt may be in a molar ratio of from 1:2 to 2:1. The salt of Formula (IV) and the metal bicarbonate salt may be in a molar ratio of from 2:3 to 3:2. The salt of Formula (IV) and the metal bicarbonate salt may be in a molar ratio of from 1:1.2 to 1.2:1. The metal bicarbonate may be sodium bicarbonate or potassium bicarbonate.

Without wishing to be bound by theory, it is thought that the salt of Formula (IV) undergoes a salt metathesis reaction with the metal bicarbonate salt to form a salt of Formula (II). The salt of Formula (II) may then be isolated from the organic solvent, e.g. via filtration followed by solvent evaporation.

Salts of Formula (IV) may be prepared by contacting a compound of Formula (I) according to the first aspect of the invention with an appropriate Brønsted acid (e.g. HCl) in solution, typically wherein the compound of Formula (I) and the Brønsted acid are in stoichiometric amounts.

The invention may be as described in one of the following numbered paragraphs:

1. A compound of Formula (I):

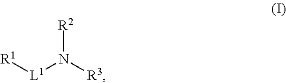

wherein:
R¹ is a dye, optionally substituted with a further L¹NR²R³ group;
L¹ is independently at each occurrence —C₁₋₁₀-alkylene-;
R² is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
L¹ and R² together with the nitrogen to which they are attached form a 3- to 6-membered heterocyclyl optionally substituted, where chemically possible, by 1 to 3 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein the heterocyclyl has 0 to 2 additional heteroatom ring vertices selected from the group consisting of O, N, and S;
R³ is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵;
R⁴ is independently at each occurrence selected from: C₃₋₆-cycloalkyl, 4- to 7-membered heterocyclyl, 5- to 10-membered heteroaryl and phenyl;
R⁵ is independently at each occurrence selected from: OR⁶, CO₂R⁶, NR⁶R⁷, CONR⁶R⁷, SR⁶, and S(O)₂NR⁶R⁷;
R⁶ is independently at each occurrence selected from H, C₁₋₆-alkyl, C₃₋₆-cycloalkyl, 4- to 7-membered heterocyclyl;
R⁷ is independently at each occurrence selected from H, C₁₋₆-alkyl, C₃₋₆-cycloalkyl, 4- to 7-membered heterocyclyl, C(O)R⁸, S(O)₂R⁸; and
R⁸ is independently at each occurrence C₁₋₆-alkyl, C₃₋₆-cycloalkyl, 4- to 7-membered heterocyclyl, 5- to 10-membered heteroaryl and phenyl;
wherein any R¹-R⁸ group that is an alkyl, alkylene, cycloalkyl, heterocyclyl, phenyl or heteroaryl may be optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl;
wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

2. A salt of Formula (II):

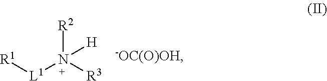

wherein:
R¹ is a dye, optionally substituted with a L¹NR²R³ group or a further L¹NH⁺R²R³·⁻OC(O)OH group;
L¹ is independently at each occurrence —C₁₋₁₀-alkylene-;
R² is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
L¹ and R² together with the nitrogen to which they are attached form a 3- to 6-membered heterocyclyl optionally substituted, where chemically possible, by 1 to 3 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R², S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein the heterocyclyl has 0 to 2 additional heteroatom ring vertices selected from the group consisting of O, N, and S;
R³ is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵;
R⁴ is independently at each occurrence selected from: C₃₋₆-cycloalkyl, 4- to 7-membered heterocyclyl, 5- to 10-membered heteroaryl and phenyl;
R⁵ is independently at each occurrence selected from: OR⁶, CO₂R⁶, NR⁶R⁷, CONR⁶R⁷, SR⁶, and S(O)₂NR⁶R⁷;
R⁶ is independently at each occurrence selected from H, C₁₋₆-alkyl, C₃₋₆-cycloalkyl, 4- to 7-membered heterocyclyl;
R⁷ is independently at each occurrence selected from H, C₁₋₆-alkyl, C₃₋₆-cycloalkyl, 4- to 7-membered heterocyclyl, C(O)R⁸, S(O)₂R⁸; and
R⁸ is independently at each occurrence C₁₋₆-alkyl, C₃₋₆-cycloalkyl, 4- to 7-membered heterocyclyl, 5- to 10-membered heteroaryl and phenyl;
wherein any R¹-R⁸ group that is an alkyl, alkylene, cycloalkyl, heterocyclyl, phenyl or heteroaryl may be optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R⁻, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl;
wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

3. A carbamate of Formula (III):

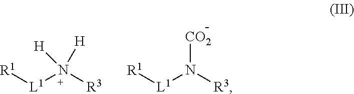

wherein:
R¹ is a dye, optionally substituted with a L¹NR²R³ group, a further L¹N⁺H₂R³ group and/or a further L¹N(CO₂⁻)R³ group;
L¹ is independently at each occurrence —C₁₋₁₀-alkylene-;
R³ is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵;
R⁴ is independently at each occurrence selected from: C₃₋₆-cycloalkyl, 4- to 7-membered heterocyclyl, 5- to 10-membered heteroaryl and phenyl;
R⁵ is independently at each occurrence selected from: OR⁶, CO₂R⁶, NR⁶R⁷, CONR⁶R⁷, SR⁶, and S(O)₂NR⁶R⁷;
R⁶ is independently at each occurrence selected from H, C₁₋₆-alkyl, C₃₋₆-cycloalkyl, 4- to 7-membered heterocyclyl;
R⁷ is independently at each occurrence selected from H, C₁₋₆-alkyl, C₃₋₆-cycloalkyl, 4- to 7-membered heterocyclyl, C(O)R⁸, S(O)₂R⁸; and
R⁸ is independently at each occurrence C₁₋₆-alkyl, C₃₋₆-cycloalkyl, 4- to 7-membered heterocyclyl, 5- to 10-membered heteroaryl and phenyl;
wherein any R¹-R⁸ group that is an alkyl, alkylene, cycloalkyl, heterocyclyl, phenyl or heteroaryl may be optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

4. The compound, salt or carbamate according to any one of paragraphs 1 to 3, wherein R¹ is a disperse dye.

5. The compound, salt or carbamate according to paragraph 4, wherein R¹ is an azo dye.

6. The compound, salt or carbamate according to paragraph 4, wherein R¹ is an anthraquinone dye.

7. The compound, salt or carbamate according to paragraph 4, wherein R¹ is selected from Disperse Red 1, Disperse Red 9, Disperse Red 17, Disperse Red 19, Disperse Red 25, Disperse Orange 3, Disperse Yellow 3, Solvent Yellow 14, Solvent Orange 7, Solvent Orange 86, Disperse Blue 3, Disperse Blue 60, Disperse Blue 87, Disperse Violet 1, and Azoic Diazo 36.

8. The compound, salt or carbamate according to any one of paragraphs 1 to 7, wherein L¹ is independently at each occurrence —C₁₋₃-alkylene-,
optionally wherein L¹ is —CH₂—.

9. The compound or salt according to any one of paragraphs 1, 2, or 4 to 9, wherein R² and R³ are each independently at each occurrence selected from:
(i) H and C₁₋₄-alkyl,
   optionally wherein R² and R³ are each independently at each occurrence selected from the group consisting of: —H, methyl, ethyl, propyl, isopropyl, butyl, isobutyl, sec-butyl, and tert-butyl; or
(ii) C₁₋₆-alkyl, C₃₋₆-cycloalkyl, 4- to 7-membered heterocyclyl, C₁₋₄-alkylene-R⁴, and C₂₋₄-alkylene-R⁵.

10. The compound or salt according to paragraph 9, wherein R² is —H and —R³ is isopropyl.

11. The compound or salt according to paragraph 9, wherein —R² and —R³ are both methyl.

12. The carbamate according to any one of paragraphs 3 to 8, wherein R³ is independently at each occurrence selected from H and C₁₋₄-alkyl,
optionally wherein R³ is independently at each occurrence selected from the group consisting of: —H, methyl, ethyl, propyl, isopropyl, butyl, isobutyl, sec-butyl, and tert-butyl.

13. A method of dyeing fibres, the method comprising the following steps:
a) subjecting the fibres to a dye liquor comprising water, and a salt of Formula (II) as paragraphed in any one of paragraphs 2 to 12, or a carbamate of Formula (III) as paragraphed in any one of paragraphs 3 to 8, and 12, or mixtures thereof, to provide the fibres wetted with the dye liquor;
b) heating the fibres wetted with the dye liquor to a temperature of from 90° C. to 180° C., to provide dyed fibres wetted with an exhausted dye liquor comprising water; and
c) removing the dyed fibres from the exhausted dye liquor.

14. The method according to paragraph 13, wherein the dye liquor of step a) is obtained by:
i) obtaining an aqueous dye liquor comprising water and a compound of Formula (I) as paragraphed in any one of paragraphs 1 to 12; and
ii) exposing the aqueous dye liquor to carbon dioxide to form the dye liquor.

15. The method according to paragraph 13 or paragraph 14, wherein the aqueous dye liquor and/or dye liquor do not comprise a dispersing agent.

16. The method according to any one of paragraphs 13 to 15, wherein the fibres are polyester fibres, optionally wherein the fibres are polyethylene terephthalate fibres.

17. The method according to any one of paragraphs 13 to 16, wherein in step b), the fibres wetted with the dye liquor are heated to a temperature of from 120° C. to 150° C. for a period of from 5 to 120 min.

18. A method of decolouring dyed fibres, wherein the fibres are dyed with a compound of Formula (I) as paragraphed in any one of paragraphs 1 to 12, the method comprising the following steps:
a) subjecting the dyed fibres to an aqueous solution;
b) exposing the dyed fibres wetted with the aqueous solution to carbon dioxide to provide decoloured fibres wetted with a decolorisation liquor, said decolorisation liquor comprising water, and a salt of Formula (II) as paragraphed in any one of paragraphs 2 to 11, or a carbamate of Formula (III) as paragraphed in any one of paragraphs 3 to 8, and 12, or mixtures thereof; and
c) removing the decoloured fibres from the decolorisation liquor

19. The method according to paragraph 18, wherein in step b), the dyed fibres wetted with the aqueous solution are exposed to carbon dioxide at a pressure of about 30 bar to 40 bar.

20. The method according to paragraph 18 or paragraph 19, wherein the dyed fibres are polyester fibres, optionally wherein the dyed fibres are polyethylene terephthalate fibres.

### BRIEF DESCRIPTION OF THE DRAWINGS

Embodiments of the invention are further described hereinafter with reference to the accompanying drawings, in which:

FIG. 1 graphically depicts the polarity of the non-ionic mixtures of the compound in water and the ionic dye in solution after exposure to carbon dioxide.

FIG. 2 graphically depicts the conversion of the ionic compound form in solution, to the non-ionic compound form in the presence of polyester, after heating above the atmospheric boil (e.g., 130° C.).

FIG. 3 graphically depicts the decarboxylation ¹HNMR experiments described herein, using Swagelok bombs.

FIG. 4 shows the formation of a reversibly switchable compound according to the invention, formed by reacting Example 1A with carbon dioxide in the presence of water.

FIG. 5 shows the stacked ¹HNMR plot of the decarboxylation of the corresponding Formula (II) salt of Example 1A, obtained using the standardised Swagelok bomb method described herein.

FIG. 6 shows the formation of a reversibly switchable compound according to the invention, formed by reacting Example 1B with carbon dioxide in the presence of water.

FIG. 7 shows the stacked ¹HNMR plot of the decarboxylation of the corresponding Formula (II) salt of Example 1B, obtained using the standardised Swagelok bomb method described herein.

FIG. 8 shows the formation of a compound according to the invention, with two reversibly switchable groups, formed by reacting Example 1C with carbon dioxide in the presence of water.

FIG. 9 shows the stacked ¹HNMR plot of the decarboxylation of the corresponding Formula (II) salt of Example 1C, obtained using the standardised Swagelok bomb method described herein.

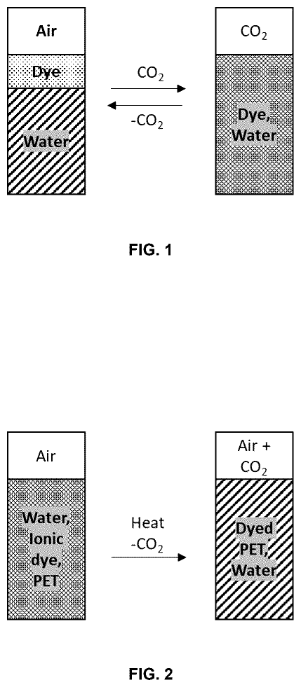

### DETAILED DESCRIPTION

The term “halo” or “halogen” refers to one of the halogens, group 17 of the periodic table. In particular the term refers to fluorine, chlorine, bromine and iodine. Preferably, the term refers to fluorine or chlorine.

The term “Cₘ₋ₙ” refers to a group with m to n carbon atoms.

The term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. “C₁₋₄ alkyl” similarly refers to such groups containing up to 4 carbon atoms. Alkylene groups are divalent alkyl groups and may likewise be linear or branched and have two points of attachment to the remainder of the molecule. Furthermore, an alkylene group may, for example, correspond to one of those alkyl groups listed in this paragraph. The alkyl and alkylene groups may be unsubstituted or substituted by one or more substituents as defined herein. Substituents for the alkyl group may be halogen, e.g. fluorine, chlorine, bromine and iodine, OH, C₁₋₄ alkoxy.

The term “C₁₋₆ alkoxy” refers to an alkyl group which is attached to a molecule via oxygen. This includes moieties where the alkyl part may be linear or branched and may contain 1, 2, 3, 4, 5 or 6 carbon atoms, for example methyl, ethyl, n-propyl, iso-propyl, n-butyl, sec-butyl, tert-butyl, n-pentyl and n-hexyl. Therefore, the alkoxy group may be methoxy, ethoxy, n-propoxy, iso-propoxy, n-butoxy, sec-butoxy, tert-butoxy, n-pentoxy and n-hexoxy. “C₁₋₄ alkoxy” similarly refers to such groups containing up to 4 carbon atoms. The alkyl part of the alkoxy group may be unsubstituted or substituted by one or more substituents. Possible substituents are described herein. Substituents for the alkyl group may be halogen, e.g. fluorine, chlorine, bromine and iodine, OH, C₁₋₆ alkoxy.

The term “C₁₋₄ haloalkyl” refers to a hydrocarbon chain substituted with at least one halogen atom independently chosen at each occurrence, for example fluorine, chlorine, bromine and iodine. The halogen atom may be present at any position on the hydrocarbon chain. For example, C₁₋₄ haloalkyl may refer to chloromethyl, fluoromethyl, trifluoromethyl, chloroethyl (e.g. 1-chloroethyl and 2-chloroethyl), trichloroethyl (e.g. 1,2,2-trichloroethyl, 2,2,2-trichloroethyl), fluoroethyl (e.g. 1-fluoroethyl and 2-fluoroethyl), trifluoroethyl (e.g. 1,2,2-trifluoroethyl and 2,2,2-trifluoroethyl), chloropropyl, trichloropropyl, fluoropropyl, and trifluoropropyl.

The term “C₂₋₄ alkenyl” refers to a branched or linear hydrocarbon chain containing at least one double bond and having 2, 3, or 4 carbon atoms. The double bond(s) may be present as the E or Z isomer. The double bond may be at any possible position of the hydrocarbon chain. For example, the “C₂₋₄ alkenyl” may be ethenyl, propenyl, butenyl, and butadienyl.

The term “C₂₋₄ alkynyl” refers to a branched or linear hydrocarbon chain containing at least one triple bond and having 2, 3, or 4 carbon atoms. The triple bond may be at any possible position of the hydrocarbon chain. For example, the “C₂₋₄ alkynyl” may be ethynyl, propynyl, and butynyl.

The term “C₃₋₆-cycloalkyl” refers to a saturated hydrocarbon ring system containing 3, 4, 5 or 6 carbon atoms. For example, the “C₃₋₆ cycloalkyl” may be cyclopropyl, cyclobutyl, cyclopentyl, cyclohexyl, bicyclo[2.1.1]hexane or bicyclo[1.1.1]pentane.

The term “heterocyclyl”, “heterocyclic” or “heterocycle” means a non-aromatic saturated or partially saturated monocyclic or fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from: nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles comprising at least one nitrogen in a ring position include, for example, azetidinyl, pyrrolidinyl, piperidinyl, piperazinyl, morpholinyl, thiomorpholinyl, tetrahydrotriazinyl, tetrahydropyrazolyl, tetrahydropyridinyl, homopiperidinyl, homopiperazinyl, 3,8-diaza-bicyclo[3.2.1]octanyl, 8-aza-bicyclo[3.2.1]octanyl, 2,5-Diaza-bicyclo[2.2.1]heptanyl and the like. Typical sulfur containing heterocycles include tetrahydrothienyl, dihydro-1,3-dithiol, tetrahydro-2H-thiopyran, and hexahydrothiepine. Other heterocycles include dihydro oxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O), for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyl groups containing 1, 2 or 3 heteroatoms selected from: nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4th Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane, and quinuclidine.

By “spiro bi-cyclic ring systems” is meant that the two ring systems share one common spiro carbon atom, i.e. the heterocyclic ring is linked to a further carbocyclic or heterocyclic ring through a single common spiro carbon atom. Examples of spiro ring systems include 3,8-diaza-bicyclo[3.2.1]octane, 2,5-Diaza-bicyclo[2.2.1]heptane, 6-azaspiro[3.4]octane, 2-oxa-6-azaspiro[3.4]octane, 2-azaspiro[3.3]heptane, 2-oxa-6-azaspiro[3.3]heptane, 6-oxa-2-azaspiro[3.4]octane, 2,7-diaza-spiro[4.4]nonane, 2-azaspiro[3.5]nonane, 2-oxa-7-azaspiro[3.5]nonane and 2-oxa-6-azaspiro[3.5]nonane.

“C₁₋₄ alkylene-C₃₋₆-cycloalkyl” means a cycloalkyl group covalently attached to a C₁₋₄ alkylene group, both of which are defined herein.

“C₁₋₄ alkylene-4- to 7-membered heterocyclyl” means a heterocyclyl group covalently attached to a C₁₋₄ alkylene group, both of which are defined herein.

“C₁₋₄ alkylene-5- to 10-membered heteroaryl” means a heteroaryl group covalently attached to a C₁₋₄ alkylene group, both of which are defined herein. Examples of heteroaralkyl groups include pyridin-3-ylmethyl and the like. The “-” in these and other groups described herein indicates the point of attachment to the remainder of the molecule.

The term “optionally substituted” refers to either groups, structures, or molecules that are substituted and those that are not substituted.

Where optional substituents are chosen from “one or more” groups set out in a list it is to be understood that this definition includes all substituents being chosen from one of the specified groups or the substituents being chosen from two or more of the specified groups in the list.

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

The term “aromatic” when applied to a substituent as a whole means a single ring or polycyclic ring system with 4n+2 electrons in a conjugated π system within the ring or ring system where all atoms contributing to the conjugated π system are in the same plane.

The term “aryl” refers to an aromatic hydrocarbon ring system. The ring system has 4n+2 electrons in a conjugated π system within a ring where all atoms contributing to the conjugated π system are in the same plane. For example, the “aryl” may be phenyl and naphthyl. The aryl system itself may be substituted with other groups.

The term “heteroaryl” refers to an aromatic mono- or bicyclic ring incorporating one or more (for example 1-4, particularly 1, 2 or 3) heteroatoms selected from: nitrogen, oxygen or sulfur. The ring or ring system has 4n+2 electrons in a conjugated π system where all atoms contributing to the conjugated π system are in the same plane.

Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from: nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl and imidazo[1,2-b][1,2,4]triazinyl. Examples of heteroaryl groups comprising at least one nitrogen in a ring position include pyrrolyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, indolyl, isoindolyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl and pteridinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from: nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

Particular examples of bicyclic heteroaryl groups containing a six membered ring fused to a five membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl, pyrrolopyridine, and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

A “-” in a substituent group denotes the point of attachment of that substituent to the rest of the molecule. For example, a —CONR⁶R⁷ is attached via the carbon of the carbonyl group.

For the absence of doubt, any divalent radical group (i.e. having two points of connection), whether depicted as a structure, e.g.

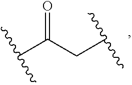 [SMILES: *CC(*)=O]

or as text, e.g. —C₁₋₅-alkylene-X—C₀₋₅-alkylene-, is positioned in the compound, salt or carbamate of Formula (I), (II), (III) or (IV) with the left and right-hand sides of the divalent radical group attached to the left and right hand portions of the rest of the compound, salt or carbamate, respectively, with the “left” and “right” being that of the reader viewing the divalent radical group and the compound, salt or carbamate as depicted in the application. In particular, for L¹ groups, the left-hand side of the depicted divalent radical is attached to the R^{1a}, R^{1b}, R^{1c} or R^{1d} group as appropriate and the right hand end is attached to the nitrogen of Formula (I), (II), (II) or (IV). For example, compounds of Formula (I) wherein L¹ is

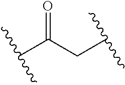 [SMILES: *CC(*)=O]

have the structure

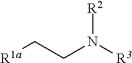

(not

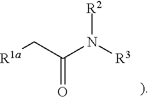

Substituents are only present at positions where they are chemically possible, the person skilled in the art being able to decide (either experimentally or theoretically) without undue effort which substitutions are chemically possible and which are not.

Reference to “about” in the context of a numerical is intended to encompass the value +/−10%. For example, about 20% includes the range of from 18% to 22%.

The term “wetted” is used in this specification to mean that the fibre substrate is in contact with a liquid, e.g. the dye liquor. It may mean that the fibre is soaked in the liquid, e.g. saturated with the liquid. It may mean that the fibre is coated in the liquid. It may mean that the fibre is immersed in the liquid.

The fibres are described as “dyed” where the compounds of the invention have absorbed onto the fibres and diffused within the fibres.

The fibres are described as “decoloured” where the compounds of the invention have been ejected from the fibres.

Azo dyes typically comprise the following group:

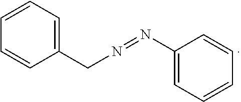 [SMILES: c1ccc(CN=Nc2ccccc2)cc1]

Azo dyes are subgrouped according to the number of azo chromophores in the molecule. Monoazo dyes contain a single azo chromophore. Diazo dyes contain two azo chromophores. Triazo dyes contain three azo chromophores.

An azo dye may a monoazo dye of Formula (VI):

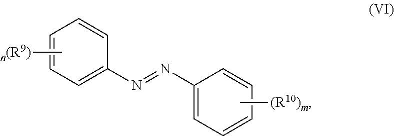

wherein R⁹ and R¹⁰ are independently at each occurrence selected from halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)₂O⁻, S(O)₂OR^{a}, S(O)R^{a}, S(O)₂R^{a}, S(O)₂NR^{a}R^{a}, CO₂R^{a}, C(O)R^{a}, CONR^{a}R^{a}, C₁-C₄-alkyl, C₂-C₄-alkenyl, C₂-C₄-alkynyl, C₁-C₄-haloalkyl and phenyl; and/or
two adjacent R⁹ groups, or two adjacent R¹⁰ groups, together with the carbon atoms to which they are attached form a phenyl or naphthyl optionally substituted, where chemically possible, by 1 to 3 substituents which are each independently selected at each occurrence from: halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)₂O⁻, S(O)₂OR^{a}, S(O)R^{a}, S(O)₂R^{a}, S(O)₂NR^{a}R^{a}, CO₂R^{a}, C(O)R^{a}, CONR^{a}R^{a}, C₁-C₄-alkyl, C₂-C₄-alkenyl, C₂-C₄-alkynyl and C₁-C₄-haloalkyl;
n is an integer from 0 to 4; and
m is an integer from 0 to 4,
wherein the sum of n and m is 1 or more,
wherein any R⁹ or R¹⁰ group that is an alkyl, alkenyl, alkynyl or phenyl group is optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)₂O⁻, S(O)₂OR^{a}, S(O)R^{a}, S(O)₂R^{a}, S(O)₂NR^{a}R^{a}, CO₂R^{a}, C(O)R^{a}, CONR^{a}R^{a},
wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

Anthraquinone dyes comprise an anthraquinone:

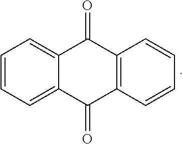 [SMILES: O=C1c2ccccc2C(=O)c2ccccc21]

Anthraquinone dyes may be of Formula (VII):

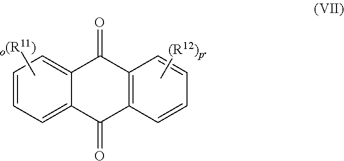

wherein R¹¹ and R¹² are independently at each occurrence selected from halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)₂O⁻, S(O)₂OR^{a}, S(O)R^{a}, S(O)₂R^{a}, S(O)₂NR^{a}R^{a}, CO₂R^{a}, C(O)R^{a}, CONR^{a}R^{a}, C₁-C₄-alkyl, C₂-C₄-alkenyl, C₂-C₄-alkynyl, C₁-C₄-haloalkyl and phenyl; and/or
two adjacent R¹¹ groups, or two adjacent R¹² groups, together with the carbon atoms to which they are attached form a 3- to 6-membered heterocyclyl optionally substituted, where chemically possible, by 1 to 3 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)₂O⁻, S(O)₂OR^{a}, S(O)R^{a}, S(O)₂R^{a}, S(O)₂NR^{a}R^{a}, CO₂R^{a}, C(O)R^{a}, CONR^{a}R^{a}, C₁-C₄-alkyl, C₂-C₄-alkenyl, C₂-C₄-alkynyl and C₁-C₄-haloalkyl;
o is an integer from 0 to 4; and
p is an integer from 0 to 4,
wherein the sum of n and m is 1 or more,
wherein any R¹¹ or R¹² group that is an alkyl, alkenyl, alkynyl or phenyl group is optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)₂O⁻, S(O)₂OR^{a}, S(O)R^{a}, S(O)₂R^{a}, S(O)₂NR^{a}R^{a}, CO₂R^{a}, C(O)R^{a}, CONR^{a}R^{a},
wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

Nitro dyes contain a nitrobenzene group:

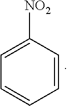

Nitro dyes may be of Formula (VIII):

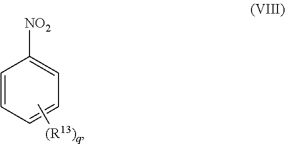

wherein R¹³ is independently at each occurrence selected from halo, nitro, cyano, NR^{a}R^{b}, NR^{a}-phenyl, NR^{a}NR^{a}S(O)₂R^{a}, NR^{a}C(O)R^{a}, NR^{a}CONR^{a}R^{a}, NR^{a}CO₂R^{a}, OR^{a}, SR^{a}, S(O)₂O⁻, S(O)₂OR^{a}, S(O)R^{a}, S(O)₂R^{a}, S(O)₂NR^{a}R^{a}, CO₂R^{a}, C(O)R^{a}, CONR^{a}R^{a}, C₁-C₄-alkyl, C₂-C₄-alkenyl, C₂-C₄-alkynyl, C₁-C₄-haloalkyl and phenyl; and/or
two adjacent R¹³ groups together with the carbon atoms to which they are attached form a phenyl or naphthyl optionally substituted, where chemically possible, by 1 to 3 substituents which are each independently selected at each occurrence from: halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)₂O⁻, S(O)₂OR^{a}, S(O)R^{a}, S(O)₂R^{a}, S(O)₂NR^{a}R^{a}, CO₂R^{a}, C(O)R^{a}, CONR^{a}R^{a}, C₁-C₄-alkyl, C₂-C₄-alkenyl, C₂-C₄-alkynyl and C₁-C₄-haloalkyl;
q is an integer from 0 to 5,
wherein any R¹¹ group that is, or forms part of an alkyl, alkenyl, alkynyl or phenyl group is optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)₂O⁻, S(O)₂OR^{a}, S(O)R^{a}, S(O)₂R^{a}, S(O)₂NR^{a}R^{a}, CO₂R^{a}, C(O)R^{a}, CONR^{a}R^{a},
wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### EXAMPLES

Decarboxylation ¹HNMR Experiments: To a 10 mL volumetric flask D₂O (10 mL), dimethyl sulfone (MSM, used as an internal standard, 10 mg, 1.1 mM), and dye (1.5 mM) were added. Samples were weighed on a semi-micro analytical balance, accurate to 5 d.p. The solution was subjected to CO₂ (0.3 L/min) whilst stirring for 1 h. The solution was then filtered under gravity, 3.125 mL was added to each of 3 Swagelok bombs and the remainder used for the baseline NMR. The bombs were then placed in the middle of an oven (130° C.) for 20, 40, and 60 min. Upon completion, samples were extracted via syringe and placed in an NMR tube for immediate analysis (see, FIG. 3). Each experiment was performed in triplicate and the concentration values obtained averaged.

### Example 1—Synthesis of Dye Compounds

### Example 1A—2-[N-ethyl-4-[(4-nitrophenyl)diazenyl]anilino]N-isopropylamine

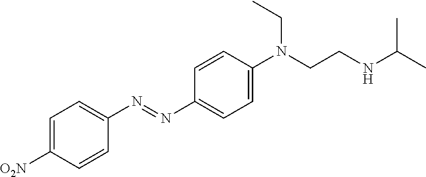 [SMILES: CCN(CCNC(C)C)c1ccc(/N=N/c2ccc([N+](=O)[O-])cc2)cc1]

2-[N-ethyl-4-(4-nitrophenyl)azoanilino]ethanol (3.143 g, 10 mmol), 4-Methylbenzene-1-sulfonyl chloride (2.67 g, 14 mmol), and N,N-diethylethanamine (2.90 ml, 20 mmol) were dissolved in acetonitrile (50 ml). The resulting red suspension was stirred at room temperature for 24 hours, after which thin layer chromatography analysis (methylene chloride) showed 2-[N-ethyl-4-(4-nitrophenyl)azoanilino]ethanol to be fully consumed. An excess of propan-2-amine (1.70 ml, 20 mmol) and potassium carbonate (2.76 g, 20 mmol) were added, and the reaction heated at reflux for a further 24 hours. The reaction underwent 60% conversion in this period. To neutralise potential alkylating side products the reaction mixture was basified (1 M sodium hydroxide, 10 ml), cooled to 40° C. and stirred for 30 min. The basified mixture was then reduced to ~10 ml in vacuo, it was then reacidified (2 M hydrochloric acid, 20 ml) and washed with methylene chloride (3×10 ml). The aqueous layer was then basified (2 M sodium hydroxide, 30 ml), and the product extracted into methylene chloride (3×50 ml), the organics were then combined and dried in vacuo. The crude product was purified via column chromatography (methylene chloride to methanol; 10:1). The product was then further purified by recrystallisation (chloroform:petroleum ether) to yield long needle-like red crystals 1.81 g (51% yield).

¹H NMR δ(CDCl₃, 400 MHz): 1.04 (3H, t, J=5.5 Hz), 1.21 (6H, d, J=5.7 Hz), 2.55 (2H, t, J=6.0 Hz), 2.71 (1H, m), 2.95 (2H, t, J=6.0 Hz) 3.54 (2H, t, J=5.5 Hz) 6.72 (2H, d, J=8.0 Hz), 7.84 (4H, m), 8.25 (2H, d, J=8.8 Hz). ¹³C NMR, DEPT 135 δ (CDCl₃, 125 MHz): 11.7 (CH₃), 12.3 (CH₃), 44.6 (CH), 50.8 (CH₂), 52.1 (CH₂), 111.4 (CH), 122.6 (CH), 124.7 (CH), 126.7 (CH). vₘₐₓ (FTIR) cm⁻¹: 2958, 2854, 1598, 1504. ESI: m/z Calc. 355.20083 (100.0%), found 356.20115 (100.0%) (M+H).

### Example 1B—1-[[2-(dimethylamino)ethyl]amino]-4-hydroxy-9,10-anthracenedione

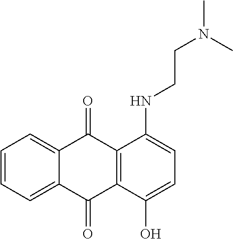 [SMILES: CN(C)CCNc1ccc(O)c2c1C(=O)c1ccccc1C2=O]

To an oven dried flask 1,4-dihydroxyanthracene-9,10-dione (3.00 g, 12.5 mmol) was added, the vessel was then purged with nitrogen for 5 minutes. N,N-Dimethylethane-1,2-diamine (2.00 ml, 18.2 mmol) was added via syringe, the resulting red solution was stirred at 55° C. for 2 hours. After 2 hours, the now purple solution was removed from nitrogen, butanol (30 ml) was added, and the solution stirred for a further 3 hours. The solution was then dried in vacuo and then purified via column chromatography (methylene chloride to methanol; 50:1) to give a purple powder 2.24 g (59% yield).

¹H NMR δ(CDCl₃, 400 MHz: 2.35 (6H, s), 2.67 (2H, t, J=6.4 Hz), 3.48 (2H, t, J=6.4 Hz), 7.25 (2H, m, 7.75 (1H, dtd, J=8.8, 7.2, 1.2 Hz), 8.35 (2H, ddd, J=8.8, 5.2, 1.2 Hz), 10.34 (1H, s), 13.69 (1H, s). ¹³C NMR, DEPT 135 δ (CDCl₃, 125 MHz): 30.9 (CH₃), 40.8 (CH₂), 58.2 (CH₂), 123.9 (CH), 126.8 (CH), 132.5 (CH), 134.2 (CH). vₘₐₓ (FTIR) cm⁻¹: 2962, 2920, 2868, 2806, 1614, 1582, 1548. ESI: m/z Calc. 310.13174 (100.0%), found 311.09134 (100.0%) (M+H).

### Example 1C—1,4-Bis[[2-(dimethylamino)ethyl]amino]-9,10-anthracenedione

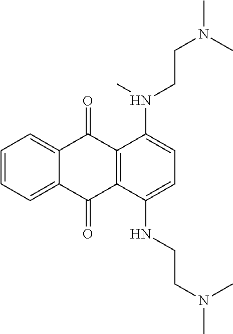 [SMILES: CN(C)CCNc1ccc(N(C)CCN(C)C)c2c1C(=O)c1ccccc1C2=O]

In a N₂ atmosphere, a solution of 9,10-dihydroxy-2,3-dihydro-1,4-anthracenedione (1 eq), N,N-dimethylethane-1,2-diamine (5 eq), and acetonitrile (10 eq) in dry toluene was heated under reflux for 24 hours. The reaction was cooled to 60° C. and diluted with ethanol, a stream of air was then passed over the surface. After 24 hours the solvents were removed in vacuo, yielding the crude product as a blue powder. The crude product was recrystallised from hexane, giving the pure blue crystalline product (95% yield).

¹H NMR δ(CDCl₃, 400 MHz): 2.39 (12H, s), 2.74 (4H, t, J=6.0 Hz), 3.57 (4H, t, J=6.0 Hz), 7.31 (2H, s), 7.68 (2H, dd, J=8.0, 3.2 Hz), 8.34 (2H, dd, J=, 8.0, 3.2 Hz), 10.73 (2H, s). ¹³C NMR, DEPT 135 δ (CDCl₃, 125 MHz): 30.9 (CH₃), 41.0 (CH₂), 58.5 (CH₂), 123.5 (CH), 126.1 (CH), 132.0 (CH). vₘₐₓ (FTIR) cm⁻¹: 2966, 2860, 2792, 1586, 1546, 1512. ESI: m/z Calc. 380.22123 (100.0%), found 381.22458 (100.0%) (M+H).

### Example 1D—5-[2-(Dimethylamino)ethoxy]-2-nitro-N-phenylbenzenamine

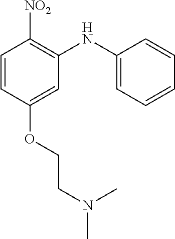 [SMILES: CN(C)CCOc1ccc([N+](=O)[O-])c(Nc2ccccc2)c1]

To a solution of 5-chloro-2-nitro-N-phenylaniline (1 eq) and 2-(dimethylamino)ethan-1-ol (10 eq) in oxolane, was added powdered potassium hydroxide (2.5 eq). Addition of potassium hydroxide resulted in a colour change from yellow to deep purple. The solution was heated under reflux for 72 hours, or until completion was determined by thin layer chromatography analysis (ethyl acetate:hexane).

The organic solvents were removed in vacuo and with aqueous hydrochloric acid (1 M), the aqueous extracts were then adjusted to pH 9 using sodium hydroxide and the solution extracted with methylene chloride. The organics were washed with brine and dried using magnesium sulphate, with the purified product being collected as a yellow oil after concentration in vacuo (yield=80%).

1H NMR (400 MHz, Chloroform-d) δ 9.77 (s, 1H), 8.21 (d, J=9.5 Hz, 1H), 7.50-7.40 (m, 2H), 7.35-7.23 (m, 3H), 6.59 (d, J=2.6 Hz, 1H), 6.38 (dd, J=9.5, 2.6 Hz, 1H), 4.03 (t, J=5.6 Hz, 2H), 2.75 (t, J=5.7 Hz, 2H), 2.36 (s, 6H). 13C NMR (125 MHz, Chloroform-d) δ 164.68, 145.61, 138.51, 129.82, 129.08, 127.54, 125.96, 124.82, 106.68, 98.08, 66.18, 57.77, 45.70. vₘₐₓ (FTIR) cm-1: 3329, 3063, 2943, 2820, 2770, 1615, 1592, 1571. ESI: m/z Calc. 301.3460 (100.0%), found 302.1498 (100.0%) (M+H).

### Example 1E—4,11-Diamino-2-(N,N-dimethylaminopropyl)naphtho[2,3-f]isoindole-1,3,5,10-tetrone (AQB-Suc-C₃)

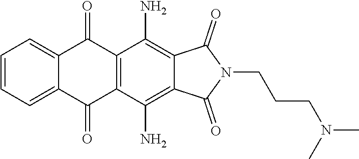 [SMILES: CN(C)CCCn1c(=O)c2c(N)c3c(=O)c4ccccc4c(=O)c3c(N)c2c1=O]

Disperse blue 60 (1.0 g, 2.6 mmol) was dissolved in 3-(dimethylamino)-1-propylamine (15 mL) and stirred for 1 hour.

The reaction mixture was dissolved in ethyl acetate (50 mL), washed with K₂CO₃ solution (3×10 mL, sat.) and extracted into formic acid solution (3×25 mL). The pH of the solution was adjusted to pH 10 using sodium carbonate solution (sat.) and extracted with chloroform (3×25 mL). The solvent was removed in vacuo, collecting the crude product as a blue solid: 0.72 g, 71%.

¹H NMR (400 MHz, CDCl₃) δ 8.27 (dd, J=5.8, 3.3 Hz, 2H), 7.78 (dd, J=5.9, 3.2 Hz, 2H), 3.72 (t, J=7.3 Hz, 2H), 2.37 (t, J=7.1 Hz, 2H), 2.24 (s, 7H), 1.87 (p, J=7.2 Hz, 2H). ¹³C NMR (101 MHz, CDCl₃) δ 184.85, 168.51, 140.68, 133.58, 133.56, 126.60, 120.17, 119.18, 56.97, 45.42, 36.26, 26.59. ESI calculated m/z: 392.15 (100.0%); found 393.1555 (100%) (M+H).

### Example 1F(1)—1-((2-(dimethylamino)ethyl)amino)anthracene-9,10-dione

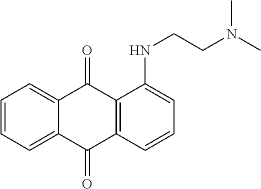 [SMILES: CN(C)CCNc1cccc2c1C(=O)c1ccccc1C2=O]

A solution of 1-chloroanthraquinone (0.97 g, 4 mmol, 1 eq) and N,N-dimethylethylenediamine (960 μL, 8.8 mmol, 2.2 eq) in toluene (5 mL) was heated under reflux for 16 hours. The reaction progress was monitored using TLC (10% methanol/DCM). The solution was cooled and extracted into formic acid (1 M, 3×2 mL), and the aqueous washes were then pH 9 using solid NaOH. The aq solution was then extracted with ethyl acetate (3×2 mL) and the extracts concentrated in vacuo to produce a red solid (0.60 g, 50% yield).

The product was further purified using chromatography (DCM, then 20% MeOH in DCM). AQR was eluted as a red solid (0.41 g, 34% yield).

¹H NMR (400 MHz, CDCl₃) δ 9.85 (s, 1H), 8.33 (dd, J=7.6, 1.5 Hz, 1H), 8.26 (dd, J=7.6, 1.5 Hz, 1H), 7.82-7.68 (m, 2H), 7.62 (dd, J=7.3, 1.3 Hz, 1H), 7.61-7.53 (m, 1H), 7.09 (dd, J=8.4, 1.4 Hz, 1H), 3.45 (td, J=6.5, 4.8 Hz, 2H), 2.71 (t, J=6.5 Hz, 2H), 2.38 (s, 6H). ¹³C NMR (125 MHz, CDCl₃) δ 184.94, 183.93, 151.55, 135.29, 135.04, 134.71, 133.91, 133.02, 132.88, 126.82, 126.68, 117.85, 115.61, 113.16, 58.04, 45.60, 41.02. ESI: m/z Calc. 294.14 (100.0%), found 295.1439 (100.0%) (M+H). Melting point 130.4-131.4° C.

### Example 1F(2)—1-((2-(dimethylamino)ethyl)amino)anthracene-9,10-dione (AQR)

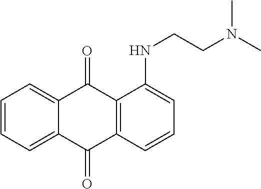 [SMILES: CN(C)CCNc1cccc2c1C(=O)c1ccccc1C2=O]

A mixture of 1-chloroanthraquinone (4.0 mmol, 1.0 g, 1.0 mol eqv.) was added to 3-(dimethylamino)-1-ethylamine (100 mmol, 11 mL, 25 mol eqv.), heated and stirred at 110° C. for 2 hours.

The reaction mixture was washed with distilled water, filtered, and dried, yielding a red powder. (3.3 mmol, 0.96 g, yield: 79%, purity by HPLC: 91 area %).

m.p.: 129-131° C. λₘₐₓ(EtOH)/nm 503 (ε/M⁻¹ cm⁻¹ 7055). vₘₐₓ/cm⁻¹ 3271, 2943, 2860, 2819, 2765, 1666, 1633, 1591, 1562 and 1504. ¹H NMR δ(CDCl₃, 400 MHz): 9.85 (s, 1H), 8.33 (ddd, J=7.8, 1.5, 0.6 Hz, 1H), 8.26 (ddd, J=7.5, 1.5, 0.5 Hz, 1H), 7.82-7.68 (m, 2H), 7.66-7.53 (m, 1H), 7.08 (dd, J=8.4, 1.3 Hz, 1H), 3.45 (td, J=6.5, 4.9 Hz, 2H), 2.70 (t, J=6.5 Hz, 2H), 2.38 (s, 6H). ¹³C NMR, DEPT 135 δ(CDCl₃, 125 MHz): 185.41 (C), 184.40 (C), 152.03 (C), 135.75 (CH), 135.51 (C), 135.18 (C), 134.38 (CH), 133.49 (C), 133.34 (CH), 127.29 (CH), 127.15 (CH), 118.33 (CH), 116.08 (CH), 113.63 (C), 58.51 (CH₂), 46.08 (CH₃), 41.50 (CH₂). HRMS: m/z calc. 294.14 (100.0%), 295.14 (19.8%), 296.14 (2.4%), found 295.14 (100.0%) (M+H).

### Example 1G—1-((3-(dimethylamino)propyl)amino)anthracene-9,10-dione (AQR-C3)

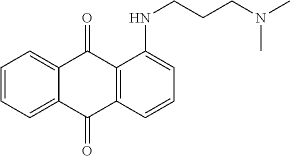 [SMILES: CN(C)CCCNc1cccc2c1C(=O)c1ccccc1C2=O]

A mixture of 1-chloroanthraquinone (6.2 mmol, 1.5 g, 1.0 mol eqv.) was added to 3-(dimethylamino)-1-propylamine (37 mmol, 4.7 mL, 6.0 mol eqv.), heated and stirred at 130° C. for 4 hours.

The product was dissolved in ethyl acetate (20 mL) and extracted into aqueous hydrochloric acid (0.10 M, 3×10 mL) from the organic layer. The pH was then adjusted to 9 using 6.0 M potassium hydroxide, and the product extracted with chloroform (3×15 mL). The organics were dried using magnesium sulphate, the solvent was removed in vacuo and the crude product was collected (4.5 mmol, 1.4 g, yield: 73%, purity by HPLC: 87 area %).

Further purification was performed via column chromatography (DCM:MeOH 9:1), obtaining a dark red waxy solid (2.9 mmol, 0.90 g, yield: 47%, purity by HPLC: 98%).

λₘₐₓ(EtOH)/nm 503 (ε/M⁻¹ cm⁻¹ 6797). ¹H NMR δ(CDCl₃, 400 MHz): 9.77 (s, 1H), 8.31-8.19 (m, 2H), 7.73 (dtd, J=23.6, 7.4, 1.4 Hz, 2H), 7.62-7.49 (m, 2H), 7.10 (dd, J=8.5, 1.3 Hz, 1H), 3.41 (td, J=7.0, 5.3 Hz, 2H), 2.47 (t, J=7.1 Hz, 2H), 2.29 (s, 5H), 1.93 (p, J=7.1 Hz, 2H). ¹³C NMR, DEPT 135 δ(CDCl₃, 125 MHz): 185.12 (C), 184.00 (C), 151.93 (C), 135.42 (CH), 135.19 (C), 134.80 (C), 134.06 (CH), 133.18 (C), 133.03 (CH), 126.80 (CH), 126.85 (CH), 118.09 (CH), 115.77 (CH), 113.08 (C), 57.24 (CH₂), 45.62 (CH₃), 41.15 (CH₂), 27.39 (CH₂). HRMS: m/z calc. 308.15 (100.0%), 309.16 (20.5%), 310.16 (2.0%), found 309.16 (100.0%) (M+H).

### Examples 1H—K

Analogous syntheses to Example 1G were performed using the following amines in replacement of 3-(dimethylamino)-1-propylamine:

| | | | Product |
|---|---|---|---|
| Amine used | Amine structure | Product name | purified yield |
| 2-(Diethylamino)-1- ethyleneamine | 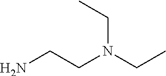 | 1-{[[1.1]2- (Diethylamino)ethyl][2.1] amino}anthracene-9,10- dione | 53% |
| 2-((4- Aminopentyl)(ethyl) amino)ethanol | 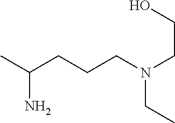 | 1-({3-[[1.1]Ethyl(2- hydroxyethyl)amino][2.1] propyl}(methyl)amino) anthracene-9,10-dione | 78% |
| 4-(2- Morpholinoethyl) aniline | 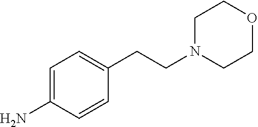 [SMILES: Nc1ccc(CCN2CCOCC2)cc1] | 1-({4-[[1.1]2-(Morpholin-4- yl)ethyl][2.1]phenyl}amino) anthracene-9,10-dione | 8% |
| 4-(2- Dimethylaminoethyl) aniline | 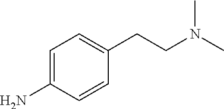 | 1-({4-[[1.1]2- (Dimethylamino)ethyl][2.1] phenyl}amino)anthracene- 9,10-dione | 9% |

### Example 1H—1-((2-(diethylamino)ethyl)amino)anthracene-9,10-dione (AQR-Et)

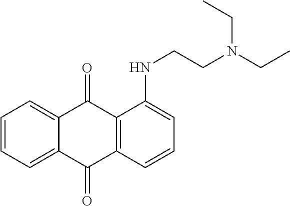 [SMILES: CCN(CC)CCNc1cccc2c1C(=O)c1ccccc1C2=O]

λₘₐₓ(EtOH)/nm 506 (ε/M⁻¹ cm⁻¹ 6930). ¹H NMR (400 MHz, CDCl₃) δ 9.82 (s, 1H), 8.34-8.27 (m, 1H), 8.27-8.20 (m, 1H), 7.72 (dtd, J=23.8, 7.4, 1.5 Hz, 2H), 7.63-7.50 (m, 2H), 7.07 (dd, J=8.4, 1.3 Hz, 1H), 3.41 (td, J=6.7, 5.0 Hz, 2H), 2.81 (t, J=6.7 Hz, 2H), 2.65 (q, J=7.1 Hz, 4H), 1.11 (t, J=7.1 Hz, 6H). ¹³C NMR, DEPT 135 δ(CDCl₃, 125 MHz): 184.77 (C), 184.00 (C), 151.62 (C), 135.20 (CH), 135.11 (C), 134.73 (C), 133.89 (CH), 133.03 (C), 132.81 (CH), 126.79 (CH), 126.66 (CH), 118.00 (CH), 115.51 (CH), 113.15 (C), 51.58 (CH₂), 47.23 (CH₂), 41.43 (CH₂), 11.94 (CH₃). HRMS: m/z calc. 322.17 (100.0%), 323.17 (21.6%), 324.17 (2.2%), found 323.18 (100.0%) (M+H).

### Example 1I—1-((3-[Ethyl(2-hydroxyethyl)amino]propyl)(methyl)amino)anthracene-9,10-dione (AQR-C4EtOH)

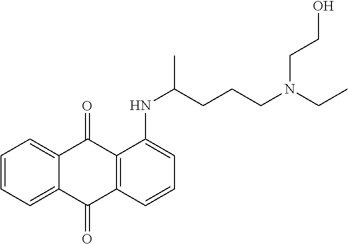 [SMILES: CCN(CCO)CCCC(C)Nc1cccc2c1C(=O)c1ccccc1C2=O]

λₘₐₓ(EtOH)/nm 509 (ε/M⁻¹ cm⁻¹ 6377). ¹H NMR (400 MHz, CDCl₃) δ 9.81 (d, J=7.8 Hz, 1H), 8.31-8.20 (m, 2H), 7.73 (dtd, J=24.4, 7.4, 1.4 Hz, 2H), 7.61-7.49 (m, 2H), 7.07 (dd, J=8.4, 1.4 Hz, 1H), 3.75 (h, J=6.1 Hz, 1H), 3.57 (t, J=5.4 Hz, 2H), 2.68-2.60 (m, 3H), 2.62-2.51 (m, 3H), 1.79-1.54 (m, 2H), 1.34 (d, J=6.4 Hz, 3H), 1.04 (t, J=7.1 Hz, 3H). ¹³C NMR, DEPT 135 δ(CDCl₃, 125 MHz): 185.01 (C), 183.85 (C), 151.21 (C), 135.34 (CH), 135.11 (C), 134.88 (C), 133.97 (CH), 133.03 (C), 132.90 (CH), 126.73 (CH), 126.64 (CH), 118.12 (CH), 115.53 (CH), 112.82 (C), 58.28 (CH₂), 55.03 (CH₂), 53.08 (CH₂), 47.97 (CH), 47.30 (CH₂), 34.60 (CH₂), 23.64 (CH₂), 20.89 (CH₃), 11.55 (CH₃). HRMS: m/z calc. 380.21 (100.0%), 381.21 (24.9%), 382.22 (2.7%), found 381.22 (100.0%) (M+H).

### Example 1J—1-({4-[2-(Morpholin-4-yl)ethyl]phenyl}amino)anthracene-9,10-dione (AQR-PhMorph)

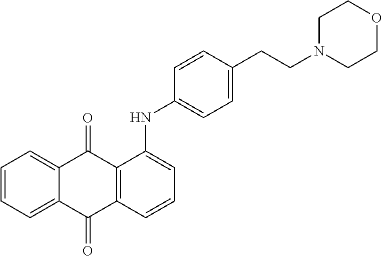 [SMILES: O=C1c2ccccc2C(=O)c2c(Nc3ccc(CCN4CCOCC4)cc3)cccc21]

¹H NMR (400 MHz, CDCl₃) δ 11.32 (s, 1H), 8.36-8.21 (m, 3H), 7.84-7.64 (m, 4H), 7.52-7.43 (m, 2H), 7.26-7.23 (m, 3H), 3.77 (t, J=4.6 Hz, 2H), 2.88-2.80 (m, 2H), 2.68-2.60 (m, 2H), 2.59-2.53 (m, 2H), 1.26 (s, 1H), 0.85 (s, 1H). LCMS: m/z calc. 412.18 (100.0%), 413.18 (28.1%), 414.19 (2.7%), 414.19 (1.1%), found 412.90 (100.0%) (M+H).

### Example 1K—1-({4-[2-(Dimethylamino)ethyl]phenyl}amino)anthracene-9,10-dione (AQR-Ph)

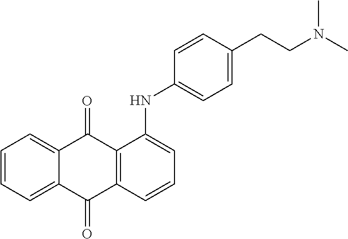 [SMILES: CN(C)CCc1ccc(Nc2cccc3c2C(=O)c2ccccc2C3=O)cc1]

¹H NMR (400 MHz, CDCl₃) δ 11.26 (s, 1H), 8.23 (dd, J=18.0, 7.5 Hz, 2H), 7.77-7.61 (m, 4H), 7.47-7.37 (m, 2H), 7.19 (s, 3H), 2.89 (t, J=8.0 Hz, 2H), 2.75 (d, J=8.8 Hz, 2H), 2.45 (s, 6H). LCMS: m/z calc. 370.17 (100.0%), 371.17 (26.0%), 372.17 (2.7%), found 370.74 (100.0%) (M+H).

### Example 2—Reversible Compound Switching in an Amine and Water System

### Example 2A—Reversible Compound Switching in a Secondary Amine Modified Azo (Example 1A) and Water System

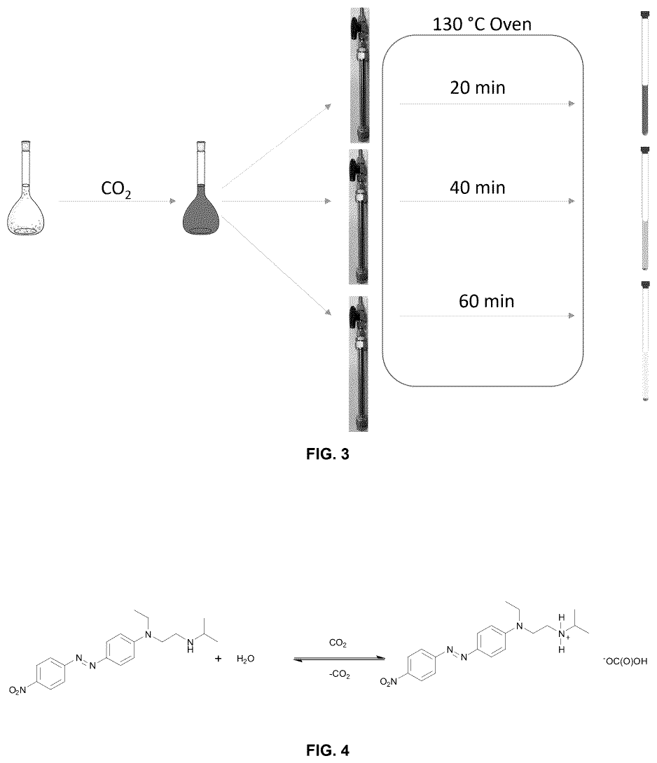

A dried sample of the compound Example 1A (1.0 mmol, see FIG. 4), internal standard dimethyl sulfone (MSM) (10 mg, 1.1 mmol) and deuterium oxide (10 mL) were placed in a dry 10 mL volumetric flask. Carbon dioxide was bubbled through the suspension via a stainless-steel syringe which was inserted into the bottom of the volumetric flask. The rate of bubbling was 0.3 L min⁻¹ for 1 hour. The suspension became increasingly red and homogeneous over time. Once the conversion was visibly completed a ¹H NMR spectrum of the resultant solution was recorded, this clearly indicated complete conversion to the corresponding Formula (II) salt of Example 1A, [(Example 1A)H][OC(O)OH](where “(Example 1A)H” is protonated Example 1A), concentration was confirmed via the internal standard. [(Example 1A)H][OC(O)OH]was attributed to the increasing concentration over time, starting from below detectable limits to fully converted quantity after completion.

[(Example 1A)H][OC(O)OH]¹H NMR δ(D₂O, 400 MHz): 1.14 (3H, t, J=5.5 Hz, Hⱼ), 1.27 (6H, d, J=5.7 Hz, Hₕ), 3.26 (2H, t, J=6.0 Hz, H_{f}), 3.41 (1H, m, H_{g}), 3.51 (2H, t, J=6.0 Hz, Hₑ) 3.74 (2H, t, J=5.5 Hz, Hᵢ) 6.89 (2H, d, J=7.2 Hz, H_{d}), 7.78 (2H, d, J=7.2 Hz, H_{c}), 7.81 (2H, d, J=7.2 Hz, Hₑ), 8.29 (2H, d, J=7.2 Hz Hₐ). vₘₐₓ (FTIR) cm⁻¹: 2958, 2854, 1598, 1504. ESI: m/z Calc. 356.20083 (100.0%), found 356.20115 (100.0%) (M+H).

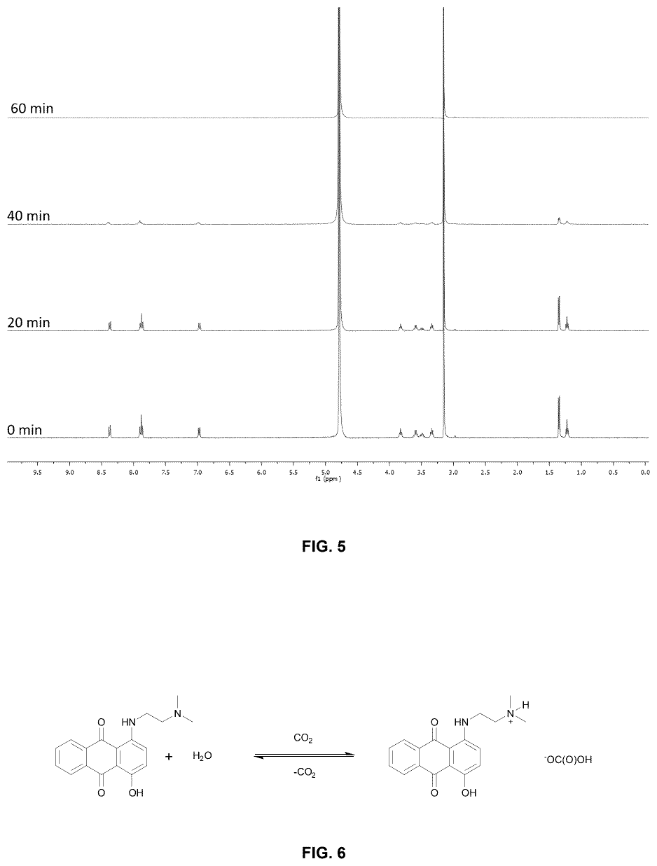

Reversibility of the reaction was confirmed when the sample was divided into three 3.125 mL portions and placed into 3 identical sealed stainless steel Swagelok tubes equipped with taps. The three tubes were heated in a 130° C. oven for 20, 40 and 60 min, respectively. The colour of the solution diminished greatly over time. The ¹H NMR spectrum after the procedure showed a reduction in concentration over time, after 60 min the spectra was consistent with trace quantities of [(Example 1A)H][OC(O)OH] and MSM in D₂O. The concentration of [(Example 1A)H][OC(O)OH]after 60 min was measured to be minimal having fallen from an initial concentration of 0.85 mmol to a final concentration of <0.01 mmol. This is evidenced in FIG. 5, wherein the stacked ¹HNMR plot shows the decarboxylation of the salt of Example 1A obtained using the standardised Swagelok bomb method described herein.

### Example 2B—Reversible Compound Switching in a Tertiary Amine Modified Anthraquinone (Example 1B) and Water System

A dried sample of the compound Example 1B (1.0 mmol, see FIG. 6), internal standard dimethyl sulfone (MSM) (10 mg, 1.1 mmol) and deuterium oxide (10 mL) were placed in a dry 10 mL volumetric flask. Carbon dioxide was bubbled through the suspension via a stainless-steel syringe which was inserted into the bottom of the volumetric flask. The rate of bubbling was 0.3 L min⁻¹ for 1 hour. The suspension became increasingly red and homogeneous over time. Once the conversion was visibly completed the ¹H NMR spectrum of the resultant solution was recorded, this clearly indicated complete conversion to the corresponding Formula (II) salt of Example 1B, [(Example 1B)H][OC(O)OH](where “(Example 1B)H” is protonated Example 1B), concentration was confirmed via the internal standard. [(Example 1B)H][OC(O)OH]was attributed to the increasing concentration over time, starting from below detectable limits to fully converted quantity after completion.

[(Example 1B)H][OC(O)OH]¹H NMR δ(D₂O, 400 MHz): 2.76 (6H, s, Hᵢ), 3.22 (2H, t, J=6.4 Hz, Hₕ), 3.48 (2H, t, J=6.4 Hz, H_{g}), 6.79 (2H, dd, J=7.2, 1.2 Hz, H_{c&f}), 7.52 (2H, m, H_{a&b}), 7.68 (2H, dd, J=, 8.8, 1.2 Hz H_{e&d}). ¹³C NMR, DEPT 135 δ(D₂O, 125 MHz): 37.2 (CH₂), 43.2 (CH₃), 55.9 (CH₂), 123.9 (CH), 125.6 (CH), 128.6 (CH), 133.2 (CH). vₘₐₓ (FTIR) cm⁻¹: 2962, 2920, 2868, 2806, 1614, 1582, 1548. ESI: m/z Calc. 310.13174 (100.0%), found 311.09134 (100.0%) (M+H).

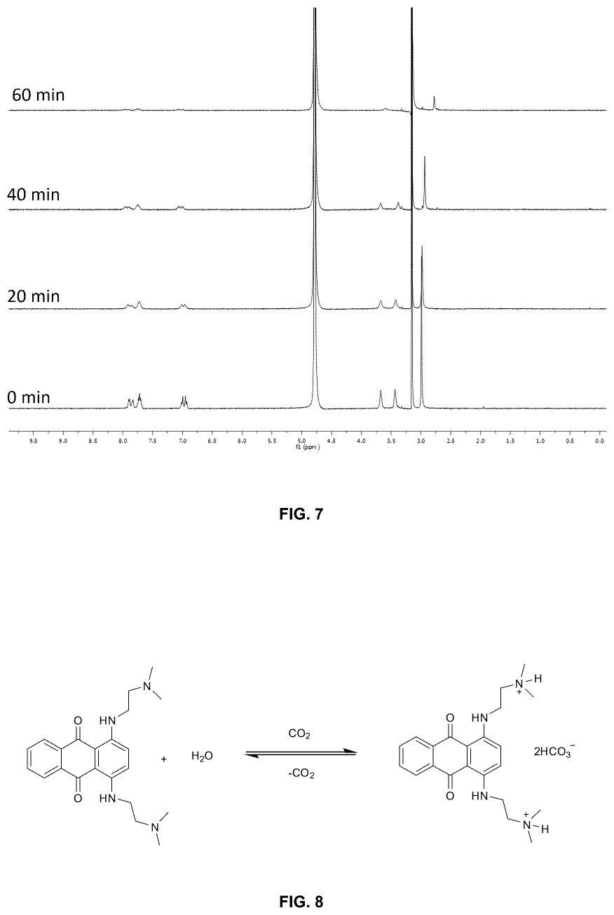

Reversibility of the reaction was confirmed when the sample was divided into three 3.125 mL portions and placed into 3 identical sealed stainless steel Swagelok tubes equipped with taps. The three tubes were heated in a 130° C. oven for 20, 40 and 60 min, respectively. The colour of the solution diminished greatly over time. The ¹H NMR spectrum after the procedure showed a reduction in concentration over time, after 60 min the spectra was consistent with trace quantities of [(Example 1B)H][OC(O)OH] and MSM in D₂O. The concentration of [(Example 1B)H][OC(O)OH]after 60 min was measured to be minimal having fallen from an initial concentration of 0.96 mmol to 0.035 mmol. This is evidenced in FIG. 7, wherein the stacked ¹HNMR plot shows the decarboxylation of the salt of Example 1B obtained using the standard method.

### Example 2C—Reversible Compound Switching in a Doubly Tertiary Amine Modified Anthraquinone (Example 1C) and Water System

A dried sample of the compound Example 1C (1.0 mmol, see, FIG. 8), internal standard dimethyl sulfone (MSM) (10 mg, 1.1 mmol) and deuterium oxide (10 mL) were placed in a dry 10 ml volumetric flask. Carbon dioxide was bubbled through the suspension via a stainless-steel syringe which was inserted to the bottom of the volumetric flask. The rate of bubbling was 0.3 L min⁻¹ for 1 hour. The suspension became increasingly homogeneous over time. Once the conversion was visibly completed a ¹H NMR spectrum of the resultant solution was recorded, this clearly indicated complete conversion to the corresponding Formula (II) salt of Example 1C [(Example 1C)H][OC(O)OH](where “(Example 1C)H” is protonated Example 1C), concentration was confirmed via the internal standard. [(Example 1C)H][OC(O)OH]was attributed to the increasing concentration over time, starting from below detectable limits to fully converted quantity after completion.

[(Example 1C)H][OC(O)OH]₂ ¹H NMR δ(D₂O, 400 MHz): 2.98 (12H, s, Hᵢ), 3.48 (4H, t, J=5.6 Hz, Hₕ), 3.83 (4H, t, J=5.6 Hz, H_{g}), 6.97 (2H, s, H_{a&b}), 7.74 (2H, dd, J=, 8.8, 1.2 Hz, H_{c&f}), 7.95 (2H, dd, J=, 8.8, 1.2 Hz, H_{e&d}). ¹³C NMR, DEPT 135 δ (D₂O, 125 MHz): 37.4 (CH₂), 43.3 (CH₃), 56.4 (CH₂), 123.8 (CH), 125.6 (CH), 132.9 (CH). vₘₐₓ (FTIR) cm⁻¹: 2966, 2860, 2792, 1586, 1546, 1512. ESI: m/z Calc. 380.22123 (100.0%), found 381.22458 (100.0%) (M+H).

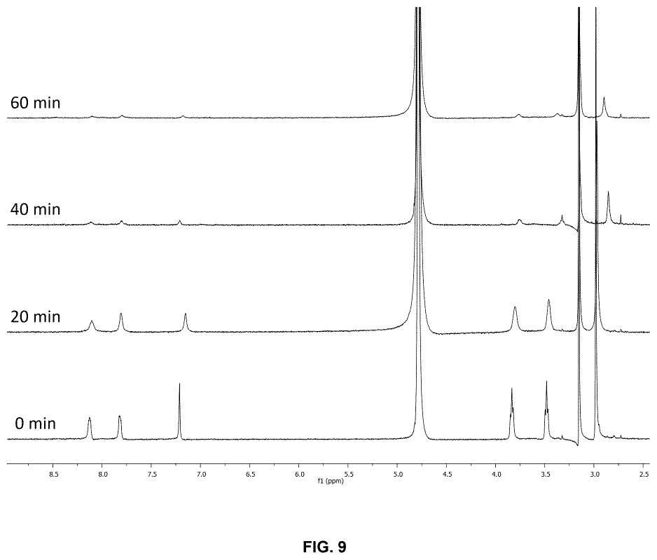

Reversibility of the reaction was confirmed when the sample was divided into three 3.125 mL portions and placed into 3 identical sealed stainless steel Swagelok tubes equipped with taps. The three tubes were heated in a 130° C. oven for 20, 40, and 60 min respectively. The colour of the solution diminished greatly over time. The ¹H NMR spectrum after the procedure showed a reduction in concentration over time, after 60 min the spectra was consistent with trace quantities of [(Example 1C)H][OC(O)OH] and MSM in D₂O. The concentration of [(Example 1C)H][OC(O)OH]after 60 min was measured to be minimal having fallen from an initial concentration of 1.03 mmol to a final concentration of 0.18 mmol. This is evidenced in FIG. 9, wherein the stacked ¹HNMR plot shows the decarboxylation of the salt of Example 1C obtained using the standardised Swagelok bomb method described herein.

### Example 3

### Example 3A—Dispersant Free Dyeing of Polyester Using a Switchable Secondary Amine Modified Azo (Example 1A) in Water

### Dyeing the Polyester

For the dyeing of polyester (1 g) at 130° C. using 1% omf (on mass of fibre) solution, with a dye liquor ratio of 50:1, Example 1A (10 mg, 0.028 mmol) was added to a 50 mL volumetric flask. To this deionised water (35 mL) was added with a small magnetic stir bar. A stainless-steel needle was inserted, reaching to the bottom of the bottom flask, through which a flow of carbon dioxide (0.3 L min⁻¹) was continued, whilst stirring, until a homogeneous solution had formed, following this additional water was added (15 mL) and a flow of CO₂ was added for a further 15 min (0.1 L min⁻¹) whilst stirring. Polyester (4×10 cm, 1 g) was rinsed by being stirred in a beaker containing distilled water (150 mL), this was removed and dried for 30 min in a 50° C. oven. The dried PET sample was then placed in a Roaches A1 180 mL high temperature dyeing process beaker with the ionic compound solution.

Dyeing beakers were placed in a Roaches infrared Pyrotec 2000 dyeing machine, samples were heated at a rate of 1.5° C. min⁻¹ to 130° C. Samples were held at 130° C. for 60 min before being cooled rapidly at a rate of 3° C. min⁻¹ to 40° C., at which temperature it was held for 10 min. The dyed samples were then rinsed by stirring in a beaker containing distilled water (150 mL), samples were then dried in a 50° C. oven for 60 min.

### Acid Clearing

The dyed fabric was then returned to the dyeing beaker in a solution of formic acid (2%) and heated in the Pyrotec dyeing machine at 70° C. for 60 min, heated at a rate of 1.5° C. min⁻¹ to 70° C., cooled at a rate of 3° C. min⁻¹ to 40° C. The dyed samples were then rinsed by stirring in a beaker containing distilled water (150 mL), samples were then dried in a 50° C. oven for 60 min. Subsequent wash fastness tests (ISO 105-C06-2010, test C2S) and spectrophotometric analysis of the samples via K/S and CIELab, showed the process to be equivalent to the reductive clearing process and gave very good to excellent results.

### Example 3B—Dispersant Free Dyeing of Polyester Using a Switchable Tertiary Amine Modified Anthraquinone (Example 1B) in Water

### Dyeing the Polyester

The process as outlined in Example 3A was repeated with the tertiary amine modified anthraquinone compound, Example 1B.

### Acid Clearing

Following the process outlined in Example 3A, Example 1B gave an average reflectance λₘₐₓ of 3.81% at 560 nm, corresponding to an average K/S value of 12.2.

### Example 4—Salt Metathesis Dyeing Method

Example 1F (100 mg, 0.33 mmol) was dissolved in dry ethanol (5 ml). The solution was acidified using conc. HCl (in a molar ratio of 1:1) and heated to 40° C. for 20 min. The solid (Example 1F·HCl) was collected by filtration under reduced pressure and then washed three times with diethyl ether (90 mg, 0.27 mmol) 82% yield. 1H NMR (400 MHz, D2O-salt) δ 7.77 (dd, J=20.9, 6.6 Hz, 2H), 7.67 (dd, J=12.8, 6.3 Hz, 2H), 7.28 (t, J=8.0 Hz, 1H), 7.09 (d, J=7.3 Hz, 1H), 6.82 (d, J=8.5 Hz, 1H), 3.58 (t, J=6.1 Hz, 2H), 3.39 (t, J=6.1 Hz, 2H), 2.96 (s, 6H).

The solid (90 mg, 0.27 mmol) was then dissolved in methanol with sodium bicarbonate in a molar ratio of 1:1 under reflux conditions. After 1 h, the solution was filtered through celite, and the solvent removed under reduced pressure. The resultant solid was washed three times with diethyl ether which afforded a red solid, i.e. Example 1F. HCO₃⁻. (32 mg, 0.16 mmol), yield 27%. 1H NMR (400 MHz, D2O-salt) δ 7.94-7.75 (m, 2H), 7.75-7.61 (m, 2H), 7.45-7.20 (m, 1H), 7.13 (s, 1H), 6.90 (dd, J=42.7, 8.2 Hz, 1H), 3.72-3.57 (m, 1H), 3.41 (d, J=6.0 Hz, 1H), 2.96 (s, 6H).

For the dyeing of polyester (1 g) at 130° C. using 1% omf solution, with a dye liquor ratio of 12.5:1, Example 1F. HCO₃ (10 mg, 0.028 mmol) was added to a 25 mL volumetric flask. To this deionised water (12.5 mL) was added with a small magnetic stir bar and stirred at rt for 20 min. Polyester (4×10 cm, 1 g) was rinsed by being stirred in a beaker containing distilled water (150 mL), this was removed and dried for 30 min in a 50° C. oven. The dried PET sample was then placed in a Roaches A1 180 mL high temperature dyeing process beaker with the ionic compound solution.

Dyeing beakers were placed in a Roaches infrared Pyrotec 2000 dyeing machine, samples were heated at a rate of 1.5° C. min-1 to 130° C. Samples were held at 130° C. for 60 min before being cooled rapidly at a rate of 3° C. min-1 to 40° C., at which temperature it was held for 10 min. The dyed samples were then rinsed by stirring in a beaker containing distilled water (150 mL), cleared using standard procedure and then dried in a 50° C. oven for 60 min.

### Example 5—Light Fastness

The light fastness of certain compounds of the present invention was assessed according to ISO 105-B02:2014, which describes a method for determining the effect of an artificial light source, representative of natural daylight (D65), on the colour of textiles. The specimen textiles were exposed to artificial light via xenon lamp under controlled temperature and humidity, together with a series of reference materials of known photostability known as the blue wool scale. The colour fastness to light is assessed by comparing the change in colour of the test sample to that of the reference samples using the grey scale for assessing change in colour fading. The given grade is equal to the number of blue wool samples that fade at the same rate as the reference materials. This produces a 1-7 scale.

Light fastness of trialled dyes according to ISO 105-B02:2014:

| Dye | Grade |
|---|---|
| AQR | 2 |
| AQR-C3 | 3 |
| AQR-Et | 2 |
| AQR-C4EtOH | 3-4 |
| AQR-PhMorph | 4-5 |
| AQR-Ph | 4-5 |

The photostability is increased from 2 to 3 by an extra carbon separation between compounds AQR and AQR-C3. AQR-Ph, with an electron withdrawing group directly attached to the 1-amino nitrogen and a 6-carbon separation between the two nitrogen groups resulted in a significant increase in photostability. The addition of a morpholine group in AQR-PhMorph also displayed good photostability.

1. A method of dyeing fibres, the method comprising the following steps:
a) subjecting the fibres to a dye liquor comprising water to provide the fibres wetted with the dye liquor;
b) heating the fibres wetted with the dye liquor to a temperature of from 90° C. to 180° C., to provide dyed fibres wetted with an exhausted dye liquor comprising water; and
c) removing the dyed fibres from the exhausted dye liquor,
wherein the dye liquor of step a) further comprises:
i) a compound of Formula (I) and carbon dioxide: 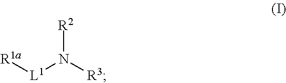
ii) a salt of Formula (II), a carbamate of Formula (III), or mixtures thereof: 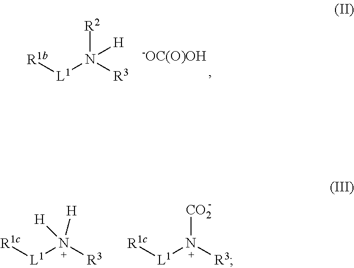
or
iii) a salt of Formula (IV) and a metal bicarbonate salt: 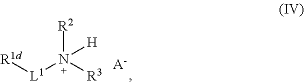
wherein:
R^{1a} is a dye, optionally substituted with a further L¹NR²R³ group;
R^{1b} is a dye, optionally substituted with a L¹NR²R³ group or a further L¹NH⁺R²R³·⁻OC(O)OH group;
R^{1c} is a dye, optionally substituted with a L¹NR²R³ group, a further L¹N⁺H₂R³ group and/or a further L¹N(CO₂⁻)R³ group;
R^{1d} is a dye, optionally substituted with a L¹NR²R³ group or a further L¹NH⁺R²R³·⁻A group;
A⁻ is an anionic counterion derived from a Brønsted acid;
L¹ is independently at each occurrence selected from —C₁₋₅-alkylene-X—C₀₋₅-alkylene- or —C₀₋₅-alkylene-X—C₁₋₅-alkylene-, wherein X is absent or selected from C₃₋₆-cycloalkylene, 4- to 7-membered heterocyclyl, 5- to 10-membered heteroaryl and phenyl;
R² is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
L¹ and R² together with the nitrogen to which they are attached form a 3- to 6-membered heterocyclyl optionally substituted, where chemically possible, by 1 to 3 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein the heterocyclyl has 0 to 2 additional heteroatom ring vertices selected from the group consisting of O, N, and S;
R³ is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
R² and R³ together with the nitrogen to which they are attached form a 3- to 6-membered heterocyclyl;
R⁴ is independently at each occurrence selected from: C₃₋₆-cycloalkyl, 4- to 7-membered heterocyclyl, 5- to 10-membered heteroaryl and phenyl;
R⁵ is independently at each occurrence selected from: OR⁶, CO₂R⁶, NR⁶R⁷, CONR⁶R⁷, SR⁶, and S(O)₂NR⁶R⁷;
R⁶ is independently at each occurrence selected from H, C₁₋₆-alkyl, C₃₋₆-cycloalkyl, 4- to 7-membered heterocyclyl;
R⁷ is independently at each occurrence selected from H, C₁₋₆-alkyl, C₃₋₆-cycloalkyl, 4- to 7-membered heterocyclyl, C(O)R⁸, S(O)₂R⁸; and
R⁸ is independently at each occurrence C₁₋₆-alkyl, C₃₋₆-cycloalkyl, 4- to 7-membered heterocyclyl, 5- to 10-membered heteroaryl and phenyl;
wherein any R¹-R⁸ group that is an alkyl, alkylene, cycloalkyl, heterocyclyl, phenyl or heteroaryl may be optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R⁸, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl;
wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

2. The method according to claim 1, wherein the dye liquor of step a) comprises water, and a salt of Formula (II), or a carbamate of Formula (III), or mixtures thereof.

3. The method according to claim 2, wherein the dye liquor of step a) is obtained by:
i) obtaining an aqueous dye liquor comprising water and a compound of Formula (I); and
ii) exposing the aqueous dye liquor to carbon dioxide to form the dye liquor.

4. The method according to claim 1, wherein the dye liquor of step a) comprises water and a salt of Formula (II) and the dye liquor of step a) is obtained by dissolving the salt of Formula (II) in water.

5. The method according to any one of claims claim 1 to 4, wherein the aqueous dye liquor and/or dye liquor do not comprise a dispersing agent.

6. The method according to any one of claims 1 to 5, wherein the fibres are polyester fibres, optionally wherein the fibres are polyethylene terephthalate fibres.

7. The method according to any one of claims 1 to 6, wherein in step b), the fibres wetted with the dye liquor are heated to a temperature of from 120° C. to 150° C. for a period of from 5 to 120 min.

8. A method of decolouring dyed fibres, wherein the fibres are dyed with a compound of Formula (I) as defined in claim 1, the method comprising the following steps:
a) subjecting the dyed fibres to an aqueous solution;
b) exposing the dyed fibres wetted with the aqueous solution to carbon dioxide to provide decoloured fibres wetted with a decolorisation liquor, said decolorisation liquor comprising water, and a salt of Formula (II) as defined in claim 1, or a carbamate of Formula (III) as defined in claim 1, or mixtures thereof; and
c) removing the decoloured fibres from the decolorisation liquor.

9. The method according to claim 8, wherein in step b), the dyed fibres wetted with the aqueous solution are exposed to carbon dioxide at a pressure of about 30 bar to 40 bar.

10. The method according to claim 8 or claim 9, wherein the dyed fibres are polyester fibres, optionally wherein the dyed fibres are polyethylene terephthalate fibres.

11. A salt of Formula (II): 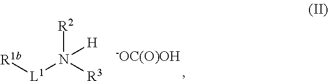
wherein:
R^{1b} is a dye, optionally substituted with a L¹NR²R³ group or a further L¹NH⁺R²R³·⁻OC(O)OH group;
L¹ is independently at each occurrence selected from —C₁₋₅-alkylene-X—C₀₋₅-alkylene- or —C₀₋₅-alkylene-X—C₁₋₅-alkylene-, wherein X is absent or selected from C₃₋₆-cycloalkylene, 4- to 7-membered heterocyclyl, 5- to 10-membered heteroaryl and phenyl;
R² is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
L¹ and R² together with the nitrogen to which they are attached form a 3- to 6-membered heterocyclyl optionally substituted, where chemically possible, by 1 to 3 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein the heterocyclyl has 0 to 2 additional heteroatom ring vertices selected from the group consisting of O, N, and S;
R³ is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
R² and R³ together with the nitrogen to which they are attached form a 3- to 6-membered heterocyclyl;
R⁴ is independently at each occurrence selected from: C₃₋₆-cycloalkyl, 4- to 7-membered heterocyclyl, 5- to 10-membered heteroaryl and phenyl;
R⁵ is independently at each occurrence selected from: OR⁶, CO₂R⁶, NR⁶R⁷, CONR⁶R⁷, SR⁶, and S(O)₂NR⁶R⁷;
R⁶ is independently at each occurrence selected from H, C₁₋₆-alkyl, C₃₋₆-cycloalkyl, 4- to 7-membered heterocyclyl;
R⁷ is independently at each occurrence selected from H, C₁₋₆-alkyl, C₃₋₆-cycloalkyl, 4- to 7-membered heterocyclyl, C(O)R⁸, S(O)₂R⁸; and
R⁸ is independently at each occurrence C₁₋₆-alkyl, C₃₋₆-cycloalkyl, 4- to 7-membered heterocyclyl, 5- to 10-membered heteroaryl and phenyl;
wherein any R¹-R⁸ group that is an alkyl, alkylene, cycloalkyl, heterocyclyl, phenyl or heteroaryl may be optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl;
wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

12. A carbamate of Formula (III): 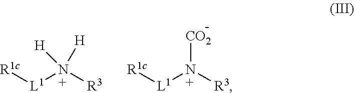
wherein:
R^{1a} is a dye, optionally substituted with a L¹NR²R³ group, a further L¹N⁺H₂R³ group and/or a further L¹N(CO₂⁻)R³ group;
L¹ is independently at each occurrence selected from —C₁₋₅-alkylene-X—C₀₋₅-alkylene- or —C₀₋₅-alkylene-X—C₁₋₅-alkylene-, wherein X is absent or selected from C₃₋₆-cycloalkylene, 4- to 7-membered heterocyclyl, 5- to 10-membered heteroaryl and phenyl;
R² is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
L¹ and R² together with the nitrogen to which they are attached form a 3- to 6-membered heterocyclyl optionally substituted, where chemically possible, by 1 to 3 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein the heterocyclyl has 1 to 2 additional heteroatom ring vertices selected from the group consisting of O, N, and S;
R³ is independently at each occurrence selected from H, C₁₋₆-alkyl, C₃₋₆-cycloalkyl, 4- to 7-membered heterocyclyl, C₁₋₄-alkylene-R⁴, and C₂₋₄-alkylene-R⁵; or
R² and R³ together with the nitrogen to which they are attached form a 3- to 6-membered heterocyclyl;
R⁴ is independently at each occurrence selected from: C₃₋₆-cycloalkyl, 4- to 7-membered heterocyclyl, 5- to 10-membered heteroaryl and phenyl;
R⁵ is independently at each occurrence selected from: OR⁶, CO₂R⁶, NR⁶R⁷, CONR⁶R⁷, SR⁶, and S(O)₂NR⁶R⁷;
R⁶ is independently at each occurrence selected from H, C₁₋₆-alkyl, C₃₋₆-cycloalkyl, 4- to 7-membered heterocyclyl;
R⁷ is independently at each occurrence selected from H, C₁₋₆-alkyl, C₃₋₆-cycloalkyl, 4- to 7-membered heterocyclyl, C(O)R⁸, S(O)₂R⁸; and
R⁸ is independently at each occurrence C₁₋₆-alkyl, C₃₋₆-cycloalkyl, 4- to 7-membered heterocyclyl, 5- to 10-membered heteroaryl and phenyl;
wherein any R¹-R⁸ group that is an alkyl, alkylene, cycloalkyl, heterocyclyl, phenyl or heteroaryl may be optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR²R², unsubstituted C₁-C₄-alkyl, unsubstituted C₂-C₄-alkenyl, unsubstituted C₂-C₄-alkynyl and unsubstituted C₁-C₄-haloalkyl; wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

13. A salt of Formula (IV): 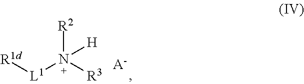
wherein:
R^{1d} is a dye, optionally substituted with a L¹NR²R³ group or a further L¹NH⁺R²R³·⁻A group;
A⁻ is an anionic counterion derived from a Brønsted acid;
L¹ is independently at each occurrence selected from —C₁₋₅-alkylene-X—C₀₋₅-alkylene- or —C₀₋₅-alkylene-X—C₁₋₅-alkylene-, wherein X is absent or selected from C₃₋₆-cycloalkylene, 4- to 7-membered heterocyclyl, 5- to 10-membered heteroaryl and phenyl;
R² is independently at each occurrence selected from H, C₁₋₅-alkyl, C₃₋₆-cycloalkyl, 4- to 7-membered heterocyclyl, C₁₋₄-alkylene-R⁴, and C₂₋₄-alkylene-R⁵; or
L¹ and R² together with the nitrogen to which they are attached form a 3- to 6-membered heterocyclyl optionally substituted, where chemically possible, by 1 to 3 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂^{a}, OR^{a}, SR^{a}, S(O)^{a}, S(O)₂R^{a}, S(O)₂NR^{a}, CO₂R^{a}, C(O)^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl; wherein the heterocyclyl has 0 to 2 additional heteroatom ring vertices selected from the group consisting of O, N, and S;
R³ is independently at each occurrence selected from H, C₁₋₆alkyl, C₃₋₆-cycloalkyl, 4- to 7-membered heterocyclyl, C₁₋₄-alkylene-R⁴, and C₂₋₄-alkylene-R⁵; or
R² and R³ together with the nitrogen to which they are attached form a 3- to 6-membered heterocyclyl;
R⁴ is independently at each occurrence selected from: C₃₋₆-cycloalkyl, 4- to 7-membered heterocyclyl, 5- to 10-membered heteroaryl and phenyl;
R⁵ is independently at each occurrence selected from: OR⁶, CO₂R⁶, NR⁶R⁷, CONR⁶R⁷, SR⁶, and S(O)₂NR⁶R⁷;
R⁶ is independently at each occurrence selected from H, C₁₋₆-alkyl, C₃₋₆-cycloalkyl, 4- to 7-membered heterocyclyl;
R⁷ is independently at each occurrence selected from H, C₁₋₆-alkyl, C₃₋₆-cycloalkyl, 4- to 7-membered heterocyclyl, C(O)R⁸, S(O)₂R⁸; and
R⁸ is independently at each occurrence C₁₋₆-alkyl, C₃₋₆-cycloalkyl, 4- to 7-membered heterocyclyl, 5- to 10-membered heteroaryl and phenyl;
wherein any R¹-R⁸ group that is an alkyl, alkylene, cycloalkyl, heterocyclyl, phenyl or heteroaryl may be optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a}, C(O)R^{a}, CONR^{a}R^{a}, unsubstituted C₁-C₄-alkyl, unsubstituted C₂-C₄-alkenyl, unsubstituted C₂-C₄-alkynyl and unsubstituted C₁-C₄-haloalkyl;
wherein R^{a} is independently at each occurrence selected from H and unsubstituted C₁-C₄-alkyl; and R^{b} is independently at each occurrence selected from H, unsubstituted C₁-C₄-alkyl, unsubstituted C(O)—C₁-C₄-alkyl, and unsubstituted S(O)₂—C₁-C₄-alkyl.

14. The method, salt, or carbamate according to any preceding claim, wherein R^{1a}, R^{1b}, R^{1c} and R^{1d} is a disperse dye.

15. The method, salt, or carbamate according to claim 14, wherein R^{1a}, R^{1b}, R^{1c} and R^{1d} is an azo dye.

16. The method, salt, or carbamate according to claim 14, wherein R^{1a}, R^{1b}, R^{1c} and R^{1d} is an anthraquinone dye.

17. The method, salt, or carbamate according to claim 14, wherein R^{1a}, R^{1b}, R^{1c} and R^{1d} is selected from Disperse Red 1, Disperse Red 9, Disperse Red 17, Disperse Red 19, Disperse Red 25, Disperse Orange 3, Disperse Yellow 3, Solvent Yellow 14, Solvent Orange 7, Solvent Orange 86, Disperse Blue 3, Disperse Blue 60, Disperse Blue 87, Disperse Violet 1, and Azoic Diazo 36.

18. The method salt, or carbamate according to any one of claims 1 to 17, wherein L¹ is independently at each occurrence —C₁₋₁₀-alkylene-.

19. The method salt, or carbamate according to any one of claims 1 to 18, wherein L¹ is independently at each occurrence —C₁₋₃-alkylene-, optionally wherein L¹ is —CH₂—.

20. The method according to any one of claims 1 to 10 and 14 to 19, or the salt according to any one of claims 11 and 13 to 19, wherein R² and R³ are each independently at each occurrence selected from:
(i) H and C₁₋₄-alkyl,
optionally wherein R² and R³ are each independently at each occurrence selected from the group consisting of: —H, methyl, ethyl, propyl, isopropyl, butyl, isobutyl, sec-butyl, and tert-butyl; and
(ii) C₁₋₆-alkyl, C₃₋₆-cycloalkyl, 4- to 7-membered heterocyclyl, C₁₋₄-alkylene-R⁴, and C₂₋₄-alkylene-R⁵.

21. The method or salt according to claim 20, wherein R² is —H and —R³ is isopropyl.

22. The method or salt according to claim 20, wherein —R² and —R³ are both methyl.

23. The method according to any one of claims 1 to 10 and 14 to 22, or the carbamate according to any one of claims 11 and 14 to 19, wherein R³ is independently at each occurrence selected from H and C₁₋₄-alkyl,
optionally wherein R³ is independently at each occurrence selected from the group consisting of: —H, methyl, ethyl, propyl, isopropyl, butyl, isobutyl, sec-butyl, and tert-butyl.

24. A method of forming a salt of Formula (II) according to any one of claims 11 and 14 to 22, a carbamate of Formula (III) according to any one of claims 12, 14 to 19 and 23, or a mixture thereof, the method comprising contacting a compound of Formula (I) as defined in any one of claims 1 and 14 to 22 with carbon dioxide.

25. A method of forming a salt of Formula (II) according to any one of claims 11 and 14 to 22, the method comprising contacting a salt of Formula (IV) according to any one of claims 13 to 22 with a metal bicarbonate salt.
